(19) United States
(12) Patent Application Publication
(10) Pub. No.: US 2003/0044772 A1
(43) Pub. Date: Mar. 6, 2003

(54) Methods for identifying ligand specific binding molecules

(75) Inventors: Jeffry D. Watkins, Encinitas, CA (US); William D. Huse, Del Mar, CA (US); Herren Wu, Houston, TX (US)

Correspondence Address: MEDLEN & CARROLL, LLP, 101 HOWARD STREET, SUITE 350, SAN FRANCISCO, CA, 94105, US

(73) Assignee: Applied Molecular Evolution [formerly IXSYS]

(21) Appl. No.: 09/977,797

(22) Filed: Oct. 15, 2001

Related U.S. Application Data
(63) Continuation of application No. 09/129,026, filed on Aug. 4, 1998, now abandoned.
(60) Provisional application No. 60/113,667, filed on Aug. 4, 1997.

(51) Int. Cl. C12Q 1/70; G01N 33/574; G01N 33/53

(52) U.S. Cl. 435/5; 435/7.23; 435/7.5

(57) ABSTRACT

The present invention provides a method for identifying a binding molecule having selective affinity for a ligand. The method consists of selectively immobilizing a diverse population of binding molecules to a solid support, simultaneously contacting the diverse population immobilized on the solid support with two or more ligands and determining at least one binding molecule which selectively binds to one or more of the ligands. The invention additionally provides a method for identifying an antibody having selective affinity for a tumor antigen. The method consists of selectively immobilizing a diverse population of antibodies to a solid support, simultaneously contacting the diverse population immobilized on the solid support with two or more tumor antigens and determining at least one antibody which selectively binds to one or more of the tumor antigens. The invention also provides an isolated binding polypeptide selective for a tumor antigen. Further provided by the present invention is a Complementarity Determining Region (CDR) or functional fragment thereof of an antibody selective for a tumor antigen.

This application claims the benefit of priority of U.S. Ser. No. 08/905,825, filed Aug. 4, 1997, which was converted to a United States Provisional Application, the entire contents of which is incorporated herein by reference.

### BACKGROUND OF THE INVENTION

The present invention relates generally to tumor cell therapy and more specifically to methods of identifying new tumor specific binding molecules and tumor specific antigens.

Recent years have provided tremendous progress in the understanding of cancer development and progression. Despite this increased knowledge, only marginal decreases in death rates from most types of cancer have been observed. The success rate of cancer therapy is increased with early diagnosis. Cancer therapy generally involves treatment with therapeutic agents that affect not only cancer cells but other cells in the body as well, often leading to debilitating side effects. Thus, identification of tumor-specific targeting agents, such as antibodies that bind to tumor antigens, would provide reagents useful for earlier diagnosis of specific types of cancers as well as tumor-specific targeting agents for cancer therapy that minimizes impact on non-tumor tissues.

Although some tumor-specific antigens have been identified, obtaining useful tumor-specific targeting agents has remained elusive. Of interest for therapeutic purposes are human antibodies that can be used to target toxins to specific types of tumors. The advantage of using human antibodies is that they are least likely to cause an immune response that would remove the antibody and toxin from the body during cancer therapy. However, development of human antibodies capable of targeting specific tumors has proven difficult.

To obtain antibodies or other agents capable of specifically targeting tumor types, a screen of a large number of possible agents is required. For example, to obtain a monoclonal antibody specific for a tumor antigen, it is necessary to generate and screen tens or hundreds of hybridoma cell lines. The process is laborious, time consuming and additionally requires the initial purification or preparation of the antigen or tumor cell.

A number of other screening approaches have now been developed, including recombinant methods utilizing bacteria and yeast, that allow identification of specific binding partners for a particular molecule of interest. For example, it is now possible to produce large display or combinatorial libraries of antibodies or other types of binding molecules. However, these methods generally require the screening of the libraries using one or more relatively purified molecules of interest.

Regardless of the available screening approaches, the identification of tumor specific antigens and their subsequent isolation has proven difficult over the years. Methods which have attempted to circumvent this problem through the use of cell lysates to screen for specific binding molecules has unfortunately resulted in molecules which exhibit inadequate binding specificities. Therefore, the availability of specific binding molecules which can be adapted to cancer diagnosis and therapy has been inherently lacking.

Thus, there exists a need for rapid and efficient methods to identify specific binding molecules to tumor antigens. The present invention satisfies this need and provides related advantages as well.

### SUMMARY OF THE INVENTION

The present invention provides a method for identifying a binding molecule having selective affinity for a ligand. The method consists of selectively immobilizing a diverse population of binding molecules to a solid support, simultaneously contacting the diverse population immobilized on the solid support with two or more ligands and determining at least one binding molecule which selectively binds to one or more of the ligands. The invention additionally provides a method for identifying an antibody having selective affinity for a tumor antigen. The method consists of selectively immobilizing a diverse population of antibodies to a solid support, simultaneously contacting the diverse population immobilized on the solid support with two or more tumor antigens and determining at least one antibody which selectively binds to one or more of the tumor antigens. The invention also provides an isolated binding polypeptide selective for a tumor antigen. Further provided by the present invention is a Complementarity Determining Region (CDR) or functional fragment thereof of an antibody selective for a tumor antigen.

### BRIEF DESCRIPTION OF THE DRAWINGS

FIG. 1 shows the optimization of human Fab binding molecule immobilization on a solid support.

FIG. 2 shows the identification of human Fab binding molecules to tumor antigens using binding molecules selectively immobilized on a solid support and compares detection efficiencies to binding molecules directly immobilized on a solid support.

FIG. 3 shows the increased binding specificity and decreased background of detection using selective immobilization of human Fab binding molecule populations on a solid support compared to direct immobilization.

FIG. 4 shows the binding specificity of human Fab binding molecules identified by selective immobilization on a solid support to tumor cell monolayers.

FIG. 5 shows fluorescent activated cell sorting of tumor cells with human Fab binding molecules identified by selective immobilization on a solid support.

### DETAILED DESCRIPTION OF THE INVENTION

The invention provides rapid and efficient methods for the identification of binding molecules which exhibit selective affinity for one or more ligands of interest. The methods are advantageous in that they allow the simultaneous screening of multiple binding molecules against multiple ligands of interest. Moreover, very little information is required regarding the identity or function of either the binding molecule or the ligand. For example, diverse populations of binding molecules can be simultaneously screened against diverse populations of ligands to rapidly identify numerous molecules exhibiting a desired binding specificity. The methods of the invention can therefore be advantageously applied for the discovery of specific reagents for diagnosis and treatment of human diseases.

As used herein, the term “binding molecule” is intended to refer to a molecule of sufficient size and complexity so as to be capable of selectively binding a ligand. Such molecules are generally macromolecules, such as polypeptides, nucleic acids, carbohydrate or lipid. However, derivatives, analogues and mimetic compounds as well as small organic compounds are also intended to be included within the definition of this term. The size of a binding molecule is not important so long as the molecule exhibits or can be made to exhibit selective binding activity to a ligand. For example, a binding molecule can be as little as about one or two, and as many as tens or hundreds of monomer building blocks which constitute a macromolecule binding molecule. Similarly, an organic compound can be a simple or complex structure so long as selective binding affinity can be exhibited.

Binding molecules can include, for example, antibodies and other receptor or ligand binding polypeptides of the immune system. Such other molecules of the immune system include for example, T cell receptors (TCR), major histocompatibility complex (MHC), CD4 receptor, and CD8 receptor. Additionally, cell surface receptors such as integrins, growth factor receptors and cytokine receptors, as well as cytoplasmic receptors such as steroid hormone receptors are substantially also included within the definition of the term binding molecule. Furthermore, DNA binding polypeptides such as transcription factors and DNA replication factors are likewise included within the definition of the term binding molecule. Finally, polypeptides, nucleic acids and chemical compounds such as those selected from random and combinational libraries are also included within the definition of the term so long as such a molecule exhibits or can be made to exhibit selective binding activity toward a ligand.

As used herein, the term “polypeptide” when used in reference to a binding molecule or a ligand is intended to refer to peptide, polypeptide or protein of two or more amino acids. The term is similarly intended to refer to derivatives, analogues and functional mimetics thereof.

As used herein, the term “ligand” refers to a molecule that can be selectively bound by a binding molecule. A ligand can be essentially any type of molecule such as polypeptide, nucleic acid, carbohydrate, lipid, or any organic derived compound. Those skilled in the art know what is meant by the meaning of the term ligand. Specific examples of ligands are the tumor antigens described herein which are selectively bound by the human antibody binding molecules described in the examples.

As used herein, the term “diverse population”is intended to refer to a group of two or more different molecules. A diverse population of binding molecules can have similar biochemical function as long as the function or structure of the binding molecules are not identical. A diverse population can include, for example, a population of binding molecules that are antibodies capable of recognizing the same or different ligands. Moreover, the same binding molecule can recognize two different ligands based on different ligand conformations. In such cases, the binding molecules would be considered distinct based on function and multiple molecules of the same binding molecule would therefore comprise a diverse population.

As used herein, the term “selective” or “selectively” when referring to the binding of a binding molecule to a ligand or the immobilization of a population to a solid support is intended to mean that the interaction can be discriminated from unwanted or non-specific interactions. Discrimination can be based on, for example, affinity or avidity and therefore can be derived from multiple low affinity interactions or a small number of high affinity interactions. For example, a binding molecule interaction with a ligand is generally greater than about 10⁻⁴ M, is preferably greater than about 10⁻⁵ M and more preferably greater than about 10^{31 6} M. High affinity interactions are generally greater than about 10⁻⁸ M to 10^{31 9} M or greater.

As used herein, the term “immobilizing” or grammatical equivalents thereof, refers to the attachment, as through the binding of a population of binding molecules, to a solid support. Immobilization can be through specific interactions with the binding molecule and an agent on the solid support. The agent can be, for example, a chemical moiety which allows covalent or non-covalent interactions sufficient to hold the population of binding molecules to the solid support. Immobilization can also be through tethers or linkers. Such linkers can be covalent linkers, hydrolyzable linkers, photo-labile linkers or other linkers that allow the binding molecules to be selectively attached. Linkers can also be polypeptides or other biomolecular linkers such as antibodies, lipid attachments, streptavidin, receptors, fusion polypeptides, or any biomolecule that can tether the binding molecule to the solid support. Additionally, domains of polypeptides can similarly be linkers. For example, hydrophobic domains which allow direct absorption to a plastic due to specific sequences which are molecular tags or recognition sequences can be linkers for binding polypeptides.

As used herein, the term “solid support” refers to a solid medium which is sufficiently stable so as to allow immobilization of a population of binding molecules. Solid supports can include, for example, membranes such as nitrocellulose, nylon, polyvinylidene difluoride, plastic, glass, polyacrylamide or agarose. Solid supports can also be made in essentially any size or shape so long as it supports the immobilization of a population of binding molecules. For example, the solid support can be a flat planar surface such as a natural or synthetic membrane filter or a glass slide. Alternatively, the solid support can be of various spherical shapes, including, for example, beads made of glass, polyacrylamide or agarose. Porous mediums can similarly be used as solid supports and such mediums are included within the definition of the term as used herein. Additionally, any of the solid supports can be modified, for example, to include functional chemical groups that can be used directly or indirectly for attachment of binding molecules or linkers.

As used herein, the term “antibody”refers to a polypeptide which binds to a ligand and is intended to be used consistently with its meaning within the art. The term immunoglobulin is similarly intended to fall within the scope of the meaning of the term antibody as it is known and used within the art. The polypeptide can be the entire antibody or it can be any functional fragment thereof which binds to the ligand. The meaning of the term is intended to include minor variations and modifications of the antibody so long as its function remains uncompromised. Functional fragments such as Fab, F(ab)₂, Fv, single chain Fv (scFv) and the like are similarly included within the definition of the term antibody. Such functional fragments are well known to those skilled in the art. Accordingly, the use of these terms in describing functional fragments of an antibody are intended to correspond to the definitions well known to those skilled in the art. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990), which are incorporated herein by reference.

As with the above terms used for describing antibodies and functional fragments thereof, the use of terms which reference other antibody domains, functional fragments, regions, nucleotide and amino acid sequences and polypeptides or peptides, is similarly intended to fall within the scope of the meaning of each term as it is known and used within the art. Such terms include, for example, “heavy chain polypeptide” or “heavy chain”, “light chain polypeptide” or “light chain”, “heavy chain variable region” (V_{H}) and “light chain variable region” (V_{L}) as well as the term “complementarity determining region” (CDR).

In the case where there are two or more definitions of a term which is used and/or accepted within the art, the definition of the term as used herein is intended to include all such meanings unless explicitly stated to the contrary. A specific example is the use of the term “CDR” to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987) and additionally by MacCallum et al., J. Mol. Biol. 262:732-745 (1996), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table I as a comparison. The exact residue numbers which encompass a particular CDR will vary depending on the sequence and size of the CDR. Those skilled in the art can routinely determine which residues comprise a particular CDR given the variable region amino acid sequence of the antibody.

As used herein, the term “tumor antigen” refers to a molecule that can be used as a marker to discriminate or distinguish a tumor cell from a normal cell. Such discriminatory markers can manifest themselves in various ways such as preferential expression or unique cellular or subcellular localization in a tumor cell. A tumor cell for which such antigens

TABLE I
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 52-56 | 47-58 |
| V_{H} CDR3 | 95-102 | 95-102 | 93-101 |
| V_{L} CDR1 | 24-34 | 24-34 | 30-36 |
| V_{L} CDR2 | 50-56 | 50-56 | 46-55 |
| V_{L} CDR3 | 89-97 | 89-97 | 89-96 |

¹Residue numbering follows the nomenclature of Kabat et al., supra
²Residue numbering follows the nomenclature of Chothia et al., supra
³Residue numbering follows the nomenclature of MacCallum et al., supra

preferentially mark can be any cell which undergoes abnormally frequent cell proliferation or unregulated survival. Such a cell can be a neoplastic cell which may or may not be fully characterized and classified as tumor or cancer cell.

The invention provides a method for identifying a binding molecule having selective affinity for a ligand. The method consists of: a) selectively immobilizing a diverse population of binding molecules to a solid support; b) simultaneously contacting the diverse population immobilized on the solid support with two or more ligands; and c) determining at least one binding molecule which selectively binds to one or more of the ligands.

The methods of the invention employ the selective immobilization of binding molecules to a solid support and the screening of the immobilized binding molecules for selective binding interactions against two or more ligands of interest. Selective immobilization functions to increase the sensitivity of the binding interaction being measured since initial immobilization of a selected population of molecules onto a solid support reduces non-specific binding interactions with irrelevant molecules or contaminants which can be present in the reaction. For example, the difficulty of measuring selective binding interactions increases with the complexity of the binding assay as well as the number and diversity of different species within the binding reaction. The methods of the invention circumvent such difficulties since the selective immobilization of the binding molecule population substantially reduces the number of unwanted binding interactions through the removal of irrelevant molecules within the reaction.

Methods exist in the art for generating and screening populations of binding molecules against one or more purified ligands or targets of interest. However, when populations of binding molecules are screened against populations of ligands or complex compositions such as cells or cell extracts, the identification of specific binding events is rare. The methods of the invention allow for the efficient screening of binding molecule populations against ligand populations for the rapid identification of selective binding events. The methods of the invention provide improved sensitivity and specificity of detection through the selective immobilization of the binding molecule population on a solid support.

The methods of the invention are applicable for both large and small populations of binding molecules which can be either substantially purified or enriched such as through separation of the population by affinity techniques. Alternatively, such diverse populations can be heterogeneous compositions such as those being contained within a cell, cell extract or an in vitro derived synthesis. Selective immobilization of such populations provides sufficient enrichment of the population so as to be applicable for screening with two or more ligands of interest for selective binding affinity.

The choice of the binding molecule population will depend on the type of binding molecule desired, the need and the ultimate intended use of the binding molecule. For example, populations of binding molecules can be produced or derived from essentially any source so long as the population contains at least one binding molecule which exhibits selective binding affinity for the ligand or ligand composition of interest or so long as the population is of sufficient diversity so that there is a very likely probability that the population will contain at least one molecule which will exhibit selective binding to the ligand of interest.

At least two general approaches can be used for obtaining binding molecule populations which will meet the above criteria. One approach is to generate binding molecule populations from molecules known to function as binding molecules or known to exhibit or be capable of exhibiting binding activity. A specific example of molecules known to function as binding molecules or exhibit binding activity include antibodies and other receptors of the immune repertoire. The normal function of such molecules is to bind essentially an infinite number of different antigens and ligands. Therefore, generating a diverse population of binding molecules from an antibody repertoire, for example, will allow the identification of a binding molecule against essentially any desired ligand. Numerous other binding molecule populations exist as well and are described further below.

A second approach is to generate a large population of unknown molecules. The population should be generated to contain a sufficient diversity of sequence or structure so as to contain a molecule which will bind to the ligand composition of interest. An advantage of this approach is that no prior knowledge of sequence, structure or function is required. Instead, all that is necessary is to generate a population of sufficient size and complexity so that the population will have a high probability of exhibiting a specific binding interaction to the ligand complex by chance. A specific example of such a population are random libraries of peptides, nucleic acids and small molecule compounds. Numerous other types of “random” library molecule populations exist and are described further below.

Prior knowledge of whether populations obtained by either of the approaches described above contain at least one binding molecule which exhibits selective binding affinity for the ligand or composition of interest is not necessary. Instead, all that is necessary is to produce a population sufficiently diverse for the intended need or purpose so as to likely contain a binding molecule against the ligands or ligand compositions of interest. Those skilled in the art will know what size and diversity is necessary or sufficient for the intended purpose.

As stated previously, the choice of which type of binding molecule population to use will depend on the specific need and desired use of the selected binding molecule. For example, if binding molecules having high affinity interaction are desired, then the use of a population of antibody binding molecules can be preferred. Other binding polypeptides and receptors of the immunoglobulin superfamily of receptors additionally can be used as well. Alternatively, if binding molecules are desired for receptors, for example, it may be desired to use populations of random peptide or nucleic acid sequences. Specific examples would include, for example, the use of random peptide populations to identify peptide binding molecules specific for cell surface receptors, random small molecule populations for the identification of binding molecules having selective binding affinity to cytoplasmic receptors such as steroid receptors, and random nucleic acid populations for the identification of binding molecules selective for nucleic acid binding polypeptides. Those skilled in the art will know or can determine what type of approach and what type of binding molecule population is applicable for an intended purpose and desired need.

The size and diversity of the binding molecule population to use will depend on, for example, the ligand population or ligand composition for which selective binding molecules are desired as well as the number of selective binding molecules desired, the range of desired affinities and the time frame available for identifying an appropriate binding molecule. For example, if the application of the methods of the invention are for the identification of one or a few binding molecules with selective affinity for a ligand, then a limited number of binding molecules can comprise the population to be selectively immobilized on a solid support. Such a limited number of binding molecules can be as small as between about two to ten but will typically comprise tens of molecules.

As the desired number of binding molecules to be identified increases, so does the size and diversity of the population. Similarly, as the range of desired affinities and speed of identification of binding molecules with selective affinity for a ligand increases, so does the size and diversity of the binding molecule population. Moreover, the size of the population of binding molecules will similarly increase as the number of ligands or complexity of the ligand population increases for which selective binding molecules are desired. Thus, as each of these factors increase, the size and diversity of the binding molecule population can similarly increase.

Moderate sized populations will consist of hundreds and thousands of different binding molecules within the population whereas a large sized binding molecule population will consist of tens of thousands and millions of different binding molecule species. More specifically, large and diverse populations of binding molecules for the identification of a binding molecule will contain any of about 10⁴, 10⁵, 10⁶, 10⁷, 10⁸, 10⁹, 10¹⁰, or more, different binding molecule species. One skilled in the art will know the approximate diversity of the population of binding molecules which will be sufficient to identify the desired number of binding molecules.

Recombinant libraries of binding molecules are particularly advantageous for the methods of the invention since large and diverse populations can be rapidly generated and screened with two or more ligands of interest. Binding molecule populations can be directly used from the cell extracts of library populations, for example. Methods employed for selectively immobilizing the binding molecule populations are discussed further below. Moreover, recombinant libraries of expressed polypeptide or nucleic acid products are applicable to the methods of the invention since they can be engineered in a large number of different ways so as to facilitate or directly function in the selective immobilization of the binding molecule population to a solid support. Recombinant library methods similarly allow for the production of a large number of binding molecule populations from naturally occurring repertoires which inherently contain features for selective immobilization of the population to a solid support. Nevertheless, whether recombinant or otherwise, essentially any source of binding molecule populations can be used so long as the source provides a sufficient size and diversity of different binding molecules appropriate for the intended purpose and so long as the population of binding molecules can be selectively immobilized to a solid support.

Thus, the invention provides a method of identifying a binding molecule having selective affinity for a ligand wherein the binding molecule population is produced by a recombinant library. Binding molecule populations other than recombinant libraries exist as well and can similarly be used for the screening and identification of binding molecules having selective affinity for a ligand.

A specific example of recombinant libraries as sources of binding molecule populations are phage expression libraries in which lysogenic phage cause the release of bacterially expressed binding molecule polypeptides. In another type of phage expression library, large numbers of potential binding molecules can be expressed as fusion polypeptides on the periplasmic surface of bacterial cells. In both of these examples, the expressed polypeptides are available for immobilization on a solid support. Libraries in yeast and higher eukaryotic cells exist as well and are similarly applicable in the methods of the invention. Those skilled in the art will know or can determine what type of library is applicable for a specific purpose.

The invention also provides a method for identifying a binding molecule selective for a ligand, wherein the binding molecule is identified from an antibody population.

As stated previously, there are at least several approaches from which to obtain a starting population of binding molecules to use for the identification of a molecule having selective binding affinity for a ligand. One approach is to use a population of binding molecules that normally exhibit binding function. Antibody binding molecules are amenable to such an approach since they inherently function as binding molecules and can be generated by biological and recombinant methods to exhibit binding specificities to essentially any antigen. Thus, the populations of binding molecules of the invention can be antibodies or functional fragments thereof.

Populations of antibody binding molecules can be generated by a variety of means known to those skilled in the art. For example, populations of antibody binding molecules can be generated from hybridoma technology and populations of monoclonal antibodies can be used in the methods of the invention to identify a monoclonal antibody binding molecule, for example, having selective affinity for a ligand. Alternatively, large populations of antibody binding molecules can be expressed, for example, by various recombinant methods known in the art.

Methods exist in the art to amplify, by polymerase chain reaction and other related methods known to those skilled in the art, essentially the entire antibody repertoire of a particular organism and express as a recombinant population a diverse combination of the heavy and light antibody chains or functional fragments thereof. Organisms from which antibody repertoires can be derived include, for example, human, mouse, human, rabbit, goat, chicken or any organism capable of producing antibodies. Functional fragments include, for example, Fab, Fv and CDR regions of the antibody molecules. The antibodies or functional fragments thereof can then be screened by selective immobilization onto a solid support. In the Examples described herein, a human Fab fragment expression library was used to screen for binding molecules with selective affinity for tumor antigens.

In addition to antibody repertoires as starting sources for binding molecule populations having inherent binding capabilities, the binding molecule populations can also be derived from other molecules of the immune system having known or inherent binding capabilities. For example, the binding molecules can be T cell receptors (TCR). T cell receptors contain two subunits, α and β, which are similar to antibody variable region sequences in both structure and function. In this regard, both subunits contain variable region which encode CDR regions similar to those found in antibodies (Immunology, Third Ed., Kuby, J. (ed.), New York, W. H. Freeman & Co. (1997), which is incorporated herein by reference). The CDR containing variable regions of TCRs bind to antigens presented on the cell surface of antigen-presenting cells and are capable of exhibiting binding specificities to essentially any particular antigen. As with antibody repertoires, there is sufficient TCR sequence information known to allow the amplification and recombinant engineering of essentially the entire TCR repertoire. Thus, TCRs are amenable to the methods of the invention since they inherently function as binding molecules and can be generated by biological and recombinant methods to exhibit binding specificities to essentially any antigen.

Other exemplary binding molecules of the immune system which exhibit known or inherent binding functions include receptors of the major histocompatibility complex (MHC), CD4 and CD8. MHC functions in mediating interactions between antigen-presenting cells and effector T cells. CD4 and CD8 receptors function in binding interactions between effector T cells and antigen-presenting cells. CD4 and CD8 have the additional advantage in that they exhibit similar CDR region structure as do antibodies and TCRs sequences.

Other binding molecules exhibiting known or inherent binding function which are amenable for use as starting populations in the methods of the invention include a variety of receptors such as cell surface, cytoplasmic and nuclear receptors. Specific example of each of these classes of binding molecules will be provided below. Binding molecules other than those discussed below also exist and can be used in the methods of the invention. Such other molecules are known or can be determined by those skilled in the art using the teachings provided herein.

Integrins, for example, are cell surface receptors involved in a variety of physiological processes such as cell attachment, cell migration and cell proliferation. Integrins mediate both cell-cell and cell-extracellular matrix adhesion events. Structurally, integrins consist of heterodimeric polypeptides where a single α chain polypeptide noncovalently associates with a single β chain. In general, different binding specificities are derived from unique combinations of distinct α and β chain polypeptides. For example, vitronectin binding integrins contain the αᵥ integrin subunit and include α_{V}β₃, α_{V}β₁ and α_{V}β₅, all of which exhibit different ligand binding specificities.

Binding molecule populations can also be produced from other cell surface receptors. These other cell surface receptors include growth factor or hormone receptors for ligands such as platelet derived growth factor, epidermal growth factor, insulin, insulin-like growth factor, hepatocyte growth factor, and other growth factors and hormones. In addition, cell surface receptors include cytokine receptors such as those for the interleukins and interferons.

Cytoplasmic receptors can additionally be used as the binding molecule population for use in the methods of the invention. Cytoplasmic receptors include, for example, the steroid hormone receptors such as the thyroid hormone receptor, retinoic acid receptor, glucocorticoid receptor, estrogen receptor and others. In addition, binding molecule populations can be generated from DNA or other nucleic acid binding polypeptides that exhibit known or inherent binding function in processes such as replication, transcription, processing or translation.

As described previously, the binding molecule populations can also be derived from random libraries of unknown sequences or structures. In this approach it is sufficient to produce a large and diverse library so as to exhibit a likely probability that one or more sequences within the binding molecule population will exhibit selective binding affinity to the ligand of interest. All that is necessary to practice the methods of the invention using such populations is to be able to selectively immobilize the random population of binding molecules to a solid support. Methods are described below for selectively immobilizing essentially any population of binding molecules to a solid support. Moreover, using the teachings described herein, those skilled in the art will know how to apply other methods so as to allow the selective immobilization of a population of binding molecules to a solid support.

Finally, the binding molecules can also be synthetic compounds produced by, for example, combinatorial chemistry methods known to those skilled in the art. Such combinatorial chemical libraries can be selectively immobilized onto a solid support and then screened for the identification of molecules having selective binding affinity for a ligand. Additionally, large populations of other compounds, such as natural or synthetic compound libraries are also equally applicable as a binding molecule population so long as they can be selectively immobilized onto a solid support.

The invention provides a method for identifying a binding molecule exhibiting selective affinity for a ligand from a diverse population of binding molecules. The binding molecule is identified by simultaneously contacting a diverse population of immobilized binding molecules with two or more ligands.

The methods of the invention allow for the simultaneous screening of two or more ligands of interest. Binding molecules exhibiting selective affinity can be obtained to one or more of the ligands of interest. The ligand population will be selected depending on the need and intended use of the binding molecule as well as the characteristics of the ligands or ligand compositions. For example, using the methods of the invention to selectively immobilize a population of binding molecules, selective binders can be identified to ligands as complex as tumor cells or specific tissue types as well as to simple ligand populations of two or more different species. An advantage of the method is that sources of relatively crude ligand populations can be used which facilitates the use of whole tissues, tumor cells and cell lysates as ligand sources. In addition, ligands which are not biochemically well characterized can additionally be used in the methods of the invention.

In the case where the ligand populations are complex compositions such as tissues, cells, cell lysates, and cellular or subcellular compartments, it is the individual molecules which comprise each of these complex compositions that make up the ligand population. Therefore, by choosing a complex composition such as a cell or cell lysate, for example, the ligand population inherently contains two or more ligands that are used to screen a population of binding molecules immobilized on a solid support. Identifying a binding molecule selective to at least one of the ligands within the population will yield a binding molecule selective to the complex composition itself. Thus, the methods of the invention are applicable to the efficient identification of binding molecules to, for example, tumor cells or other cellular pathologies for the selective targeting of therapeutic agents. The methods of the invention are similarly applicable to the identification of binding molecules to normal or diseased tissue for the selective targeting of, for example, diagnostic agents such as imaging reagents.

In order to identify a binding polypeptide having selective binding affinity to the ligand, it is necessary to selectively immobilize a population of binding molecules and then to contact the binding molecules with two or more ligands. As described further below, successful binding of a binding molecule within the population indicates selective binding to at least one of the ligands within the population. Selectivity can be further confirmed by, for example, comparing the binding affinity of the positive binder to a non-specific or control ligand population. If desired, such a comparison can be performed as a control during the initial identification of the binding molecule such as using normal cells and tumor cells as the control and ligand populations, respectively. Alternatively, such a comparison can be subsequently performed using either the initial ligand populations or more purified form of either the binding molecule or the ligand of interest.

Thus, the invention provides a method of identifying a binding molecule having selective affinity for a ligand wherein the ligand is a tissue or tumor antigen. The invention also provides a method of identifying a binding molecule having selective affinity for a ligand wherein the ligand is a polypeptide or other macromolecule in a cell lysate.

In addition to ligand populations derived from crude cell preparations or other complex compositions, the ligand populations can also be derived from simple populations of substantially purified molecules. For example, if it is desired to produce a binding molecule selective for any one member of a population of ligands, then each individual member can be combined into a single population and screened simultaneously using the methods of the invention. The molecules can be substantially purified or contain various amounts of other irrelevant species. Additionally, in cases such as this, where a binding molecule is desired which is selective for one or a few members of a population, if the individual ligand species can be produced by recombinant or other biological synthetic mechanisms, then cells can be engineered to express the ligands. The ligands can then be screened in crude cell extracts to identify a binding molecule which is selective for at least one ligand in the population. Such cells can be engineered to synthesize each ligand individually and then cell extracts can be pooled for screening. Alternatively, a cell can be engineered to express multiple ligands so that only one or a few cell extracts are required to be combined and screened.

The ligand populations can be composed of different sizes of either substantially purified molecules or crude cell preparations or other complex compositions. Generally, simple populations of ligands contain at least two different ligand species, and can be composed of any of 3, 4, 5, 6, 7, 8, 9 or 10 different ligands. Moderate ligand populations contain between about ten and several hundreds of different ligand species. Complex ligand populations contain about tens of thousands of different ligand species including, for example, the number of different molecules within a cell. This number is generally thought to be about 10⁵. Even further, recombinant libraries and combinatorial libraries can be screened which can be composed of ligand populations of 10⁶, 10⁷, 10⁸ and as large as greater than 10¹⁰ different species. Each of these population sizes can be used as a ligand population for which to identify a binding molecule having selective binding affinity. The choice of the population size and type will depend on the need and intended use of the binding molecule. One skilled in the art will know which size and type of population is suitable for a particular need.

As stated previously, the ligands which comprise the above described populations can be essentially any type of molecule such as a polypeptide, nucleic acid, carbohydrate, lipid or any organic compound. Therefore, it is understood that molecules discussed above as binding molecules can also be ligands. For example, an individual or group of cell surface receptors such as integrins, growth factor receptors or cytokine receptors can be used as ligands to screen a random peptide library or combinatorial chemical library to identify binding molecules that could be used as agonists or antagonists for these receptors.

To conduct a screen for binding molecules which bind to a ligand or group of ligands, the ligands themselves are detected by an appropriate detection method. The detection method can be direct or indirect and can involve detection of, for example, light emission, radioisotopes, color development, or any method which allows the ligand to be detected. Direct detection can involve methods such as radioactive labeling of ligands using metabolic labeling in an appropriate cell or in vitro labeling using in vitro coupled transcription-translation with appropriate radioactive amino acids, covalent modification with a radioactive substrate using an appropriate enzyme such as a kinase, or chemical modification using radioisotopes such as iodination. Direct methods can also involve fusion of an appropriate detection molecule to the ligand. For example, the ligand can be fused to luciferase and detected by light emission or can be fused to lacZ and detected by appropriate calorimetric detection. In the case where a population of ligands is desired to have biologically derived detection moieties, a library containing ligands fused to such detectable molecules as luciferase and lacZ can be used to screen for binding molecules. Direct detection of a ligand can also be performed by covalent modification of the ligand by incorporating a chemical moiety capable of being detected. For example, biotin can be covalently attached to the ligand and subsequently detected by steptavidin using one of the detection methods discussed above.

For indirect detection of ligands, a molecule that is not covalently attached to the ligand can be used for detection. A molecule known to interact with the ligand is used for such indirect detection methods. For example, a known molecule such as an antibody capable of specifically detecting the ligand can be employed. The known molecule is either itself detected by one of the methods described above for direct detection of the ligand or is detected by a secondary molecule specific for the first known molecule. For example, an antibody specific for a ligand can be detected using a secondary antibody capable of interacting with the first antibody specific for the ligand, again using the detection methods described above for direct detection.

In one embodiment, ligands are derived from the cell surface of tumor cells. Cell surface molecules can be labeled, for example, with a detectable moiety such as a radioisotope or biotin. This labeling provides a source of ligand where the only characteristic that needs to be known is that it is on the surface of a cell. As described in the Examples, tumor antigen ligands were prepared by labeling cell surface polypeptides of breast carcinoma cells with biotin and used to identify Fab fragment binding molecules selective for the tumor cell surface polypeptides. Ligand populations derived from other cell or subcellular compartments can similarly be used in the methods of the invention to obtain a binding molecule that exhibits selective affinity for at least one ligand within the initial population. Thus, the methods of the invention are applicable to a large variety of ligand populations in which selective binding affinity is required for the therapeutic treatment or diagnosis of a disease.

The methods of the invention consist of selectively immobilizing a diverse population of binding molecules to a solid support. For selective immobilization, either an inherent characteristic of the binding molecules which comprise the population is exploited to provide selective immobilization or, alternatively, the molecules are engineered to contain a specific characteristic to be used for selective immobilization. For example, a binding molecule itself may contain a hydrophobic chemical group or domain or may be fused to a hydrophobic chemical group or domain that causes the binding molecule to be immobilized to a hydrophobic solid support such as plastic. In another example, the solid support can be coated with a chemical moiety or a biomolecule such that it is able to bind to and selectively immobilize only the binding molecules that make up the binding molecule population. For example, the solid support can be coated with a biomolecule that selectively binds to a domain or sequence that is common to the binding molecules. The use of such biomolecules as linkers or tethers should be selected so that they do not interfere with the ligand binding to the binding molecules. For example, an antibody against the constant region of an antibody can be used to selectively immobilize antibody binding molecules. Such an approach was used to immobilize human Fab fragments with anti-human kappa antibodies as described in the Examples. Alternatively, antibodies specific to peptide tags similarly function to selectively immobilize a binding molecule population onto a solid support.

One advantage of selective immobilization is that it provides increased sensitivity and decreased non-specific binding for detecting specific binding molecules of interest. Solid supports can, for example, immobilize a high concentration of binding molecules. Such high concentrations of binding molecules can serve to increase the ability to detect low affinity interactions with ligands and to detect ligands in low concentrations. Essentially any solid support is amenable for use in the methods of the invention. The support should be chosen to fit a particular need and to exhibit the capacity to immobilize all, or a statistically representative number of binding molecules of a population. In addition, solid supports can be made of porous materials which allow greater densities of immobilized binding molecules to be achieved.

Solid supports can also be chosen to increase manipulability of a desired screen while maintaining the ability to retain the binding molecule population. Such manipulations can be important for removing unbound ligand populations and for washing to remove non-specific interactions. The ease of manipulation can be an advantage when performing high throughput screening of a large number of binding molecules.

To perform the methods of the invention, once the population of binding molecules are immobilized on a solid support, the immobilized population is simultaneously contacted with a population of two or more ligands. The population of ligands is incubated with the selectively immobilized binding molecules under appropriate conditions for an appropriate period of time so as to allow binding of the ligands to the binding molecules. A binding molecule having selective affinity is identified by determining the binding of at least one ligand within the population to at least one binding molecule. One skilled in the art will know the appropriate conditions and can readily determine conditions sufficient to allow interactions between binding molecules and ligands so as to detect the selective binding of a binding molecule to a ligand, thus allowing the identification of a binding molecule having selective affinity for a ligand.

In addition, the invention provides methods for determining the sequence of identified binding molecules. For example, if the binding molecules are produced in an expression library, encoding nucleic acids can be isolated from selected clones expressing binding molecules identified in a screen. The encoding nucleic acids can then be sequenced using methods known to those skilled in the art.

Additionally, the invention provides a method for characterizing a ligand which is selectively bound by a binding molecule. Once a binding molecule has been identified which is selective for a ligand, the ligand can then be isolated by, for example, affinity methods known to those skilled in the art, and characterized. This characterization can be beneficial if the ligands used in the screen are not well characterized. Characterization of the ligand includes such techniques as determining its apparent molecular weight by gel electrophoresis. Other methods applicable for characterizing ligands include, for example, high performance liquid chromatography (HPLC), mass spectrometry, or other methods that provide information about the physical, biochemical or functional properties of the ligand. One skilled in the art will know appropriate methods for such characterization of ligands.

The invention additionally provides a method for identifying an antibody having selective affinity for a tumor antigen. The method consists of: a) selectively immobilizing a diverse population of antibodies to a solid support; b) simultaneously contacting the diverse population immobilized on the solid support with two or more tumor antigens; and c) determining at least one antibody which selectively binds to one or more of the tumor antigens.

The methods of the invention can be practiced using antibody and tumor cells as the binding molecules and ligand populations, respectively. The population of antibody binding molecules can be produced, for example, in an expression library. Labeling of cell surface molecules of a tumor cell provides a population of detectable tumor antigen ligands.

Using the methods of the invention, the antibody binding molecules are selectively immobilized to a solid support by, for example, the use of antibodies which are specific to the constant region of antibody binding molecules or specific to a particular isotype or antibody subunit. For example, anti-kappa chain antibodies can be used to immobilize intact antibodies as well as Fab fragments to a solid support.

Antibody binding molecules immobilized to a solid support can be incubated with tumor antigens under appropriate conditions so as to allow the tumor antigens to bind to the antibody binding molecules. The antigens can be applied as whole cells, cell extracts or as specific cellular fractions. After an appropriate period of time, unbound tumor antigens are removed from the solid support and selective binding is detected as previously described. One skilled in the art will know what conditions will allow binding of tumor cell ligands to antibodies and can readily determine the conditions sufficient to allow interactions to allow identification of antibodies specific for tumor antigens. Tumor antigens are detected to determine one or more antibody binding molecules which specifically interact with tumor antigens. The selectivity of an antibody binding molecule for a tumor antigen can be determined relative to a normal cell which is used as a control. Thus, a method of the invention provides a means to identify tumor antigen-specific antibody binding molecules which can be used for diagnostic or therapeutic purposes.

Numerous antibody binding molecules selective for tumor specific ligands have been identified by the methods of the invention. These antibody binding molecules are described below in the Examples. Moreover, the nucleotide sequences have been determined for the CDR regions of these tumor selective antibody binding molecules. Such encoding nucleic acids are similarly described below in the Examples. Thus, the invention provides a method of identifying a population of human antibodies or functional fragments thereof.

Also provided herein is an isolated binding polypeptide selective for a tumor antigen. The isolated binding polypeptide is an antibody fragment selected from the group of antibody fragments consisting of F3, F13, F14, F15, F19, F21, F22, F23, F26, F30, F31, F32, F33, F34, F35, F36, F37, F38, F40, F41, F42, F46, F49, F50, F52, F54, F55, F58, F63, F66, F67, F68, F69, F70, F72, F74, F76, F78, F79, F80, F81, F84, F85, F86, F93, F99, F104, F111, F112, F118, F126, F129, F130, F132, F133, F134, F135, F136, F138, F151, F158, F160, F174, F176, F177, F184, F186, F191, F197, F200, F202, F203, F207, F208, F212, F214, F217, F224, F231, F236, F238, TA50 and TA73. Such an isolated binding polypeptide selective for a tumor antigen can be, for example, an F3, F14, F15, F19, F21, F22, F23, F26, F133, TA50or TA73 antibody fragment.

A particularly useful isolated binding polypeptide selective for a tumor antigen can be an antibody fragment having an amino acid sequence including three particular light chain CDR sequences and three particular heavy chain CDR sequences. An isolated antibody fragment selective for a tumor antigen can have, for example, an amino acid sequence including the three light chain CDR sequences SEQ ID NO:6, SEQ ID NO:28 and SEQ ID NO:50 and the three heavy chain CDR sequences SEQ ID NO:72, SEQ ID NO:94 and SEQ ID NO:116 found in F3, or can have an amino acid sequence in which one or more of these CDRs are replaced by a substantially similar or unrelated CDR. From the above description, one skilled in the art understands that an isolated antibody fragment of the invention can have any sub-combination of the six F3 CDRs, such as any two, three, four or five F3 CDRs, or can have a single F3 CDR. An isolated antibody fragment selective for a tumor antigen also can have an amino acid sequence including the three light chain CDR sequences SEQ ID NO:8, SEQ ID NO:30 and SEQ ID NO:52 and the three heavy chain CDR sequences SEQ ID NO:74, SEQ ID NO:96 and SEQ ID NO:118 found in F14, or can have an amino acid sequence in which one or more of these CDRs are replaced by a substantially similar or unrelated CDR. From the above description, one skilled in the art understands that an isolated antibody fragment of the invention can have any sub-combination of the six F14 CDRs, such as any two, three, four or five F14 CDRs, or can have a single F14 CDR.

In another embodiment, an isolated antibody fragment selective for a tumor antigen can have an amino acid sequence including the three light chain CDR sequences SEQ ID NO:10, SEQ ID NO:32 and SEQ ID NO:54 and the three heavy chain CDR sequences SEQ ID NO:76, SEQ ID NO:98 and SEQ ID NO:120 found in F15, or can have an amino acid sequence in which one or more of these CDRs are replaced by a substantially similar or unrelated CDR. From the above description, one skilled in the art understands that an isolated antibody fragment of the invention can have any sub-combination of the six F15 CDRs, such as any two, three, four or five F15 CDRs, or can have a single F15 CDR. In another embodiment, an isolated antibody fragment selective for a tumor antigen can have an amino acid sequence including the three light chain CDR sequences SEQ ID NO:12, SEQ ID NO:34 and SEQ ID NO:56 and the three heavy chain CDR sequences SEQ ID NO:78, SEQ ID NO:100 and SEQ ID NO:122 found in F19, or can have an amino acid sequence in which one or more of these CDRs are replaced by a substantially similar or unrelated CDR. From the above description, one skilled in the art understands that an isolated antibody fragment of the invention can have any sub-combination of the six F19 CDRs, such as any two, three, four or five F19 CDRs, or can have a single F19 CDR.

In another embodiment, an isolated antibody fragment selective for a tumor antigen can have an amino acid sequence including the three light chain CDR sequences SEQ ID NO:14, SEQ ID NO:36 and SEQ ID NO:58 and the three heavy chain CDR sequences SEQ ID NO:80, SEQ ID NO:102 and SEQ ID NO:124 found in F21, or can have an amino acid sequence in which one or more of these CDRs are replaced by a substantially similar or unrelated CDR. From the above description, one skilled in the art understands that an isolated antibody fragment of the invention can have any sub-combination of the six F21 CDRs, such as any two, three, four or five F21 CDRs, or can have a single F21 CDR. In another embodiment, an isolated antibody fragment selective for a tumor antigen can have an amino acid sequence including the three light chain CDR sequences SEQ ID NO:16, SEQ ID NO:38 and SEQ ID NO:60 and the three heavy chain CDR sequences SEQ ID NO:82, SEQ ID NO:104 and SEQ ID NO:126 found in F22, or can have an amino acid sequence in which one or more of these CDRs are replaced by a substantially similar or unrelated CDR. From the above description, one skilled in the art understands that an isolated antibody fragment of the invention can have any sub-combination of the six F22 CDRs, such as any two, three, four or five F22 CDRs, or can have a single F22 CDR.

In another embodiment, an isolated antibody fragment selective for a tumor antigen can have an amino acid sequence including the three light chain CDR sequences SEQ ID NO:18, SEQ ID NO:40 and SEQ ID NO:62 and the three heavy chain CDR sequences SEQ ID NO:84, SEQ ID NO:106 and SEQ ID NO:128 found in F23, or can have an amino acid sequence in which one or more of these CDRs are replaced by a substantially similar or unrelated CDR. From the above description, one skilled in the art understands that an isolated antibody fragment of the invention can have any sub-combination of the six F23 CDRs, such as any two, three, four or five F23 CDRs, or can have a single F23 CDR. In another embodiment, an isolated antibody fragment selective for a tumor antigen can have an amino acid sequence including and the three light chain CDR sequences SEQ ID NO:20, SEQ ID NO:42 and SEQ ID NO:64 and the three heavy chain CDR sequences SEQ ID NO:86, SEQ ID NO:108 and SEQ ID NO:130 found in F26, or can have an amino acid sequence in which one or more of these CDRs are replaced by a substantially similar or unrelated CDR. From the above description, one skilled in the art understands that an isolated antibody fragment of the invention can have any sub-combination of the six F26 CDRs, such as any two, three, four or five F26 CDRs, or can have a single F26 CDR.

In another embodiment, an isolated antibody fragment selective for a tumor antigen can have an amino acid sequence including and the three light chain CDR sequences SEQ ID NO:22, SEQ ID NO:44 and SEQ ID NO:66 and the three heavy chain CDR sequences SEQ ID NO:88, SEQ ID NO:110 and SEQ ID NO:132 found in F133, or can have an amino acid sequence in which one or more of these CDRs are replaced by a substantially similar or unrelated CDR. From the above description, one skilled in the art understands that an isolated antibody fragment of the invention can have any sub-combination of the six F133 CDRs, such as any two, three, four or five F133 CDRs, or can have a single F133 CDR.

In another embodiment, an isolated antibody fragment selective for a tumor antigen can have an amino acid sequence including and the three light chain CDR sequences SEQ ID NO:24, SEQ ID NO:46 and SEQ ID NO:68 and the three heavy chain CDR sequences SEQ ID NO:90, SEQ ID NO:112 and SEQ ID NO:134 found in TA50, or can have an amino acid sequence in which one or more of these CDRs are replaced by a substantially similar or unrelated CDR. From the above description, one skilled in the art understands that an isolated antibody fragment of the invention can have any sub-combination of the six TA50 CDRs, such as any two, three, four or five TA50 CDRs, or can have a single TA50 CDR.

In still another embodiment, an isolated antibody fragment selective for a tumor antigen can have an amino acid sequence including and the three light chain CDR sequences SEQ ID NO:26, SEQ ID NO:48 and SEQ ID NO:70 and the three heavy chain CDR sequences SEQ ID NO:92, SEQ ID NO:114 and SEQ ID NO:136 found in TA73, or can have an amino acid sequence in which one or more of these CDRs are replaced by a substantially similar or unrelated CDR. From the above description, one skilled in the art understands that an isolated antibody fragment of the invention can have any sub-combination of the six TA73 CDRs, such as any two, three, four or five TA73 CDRs, or can have a single TA73 CDR.

Additionally provided is a CDR or functional fragment thereof of an antibody selective for a tumor antigen, where the CDR is derived from an antibody fragment selected from the group consisting of F3, F13, F14, F15, F19, F21, F22, F23, F26, F30, F31, F32, F33, F34, F35, F36, F37, F38, F40, F41, F42, F46, F49, F50, F52, F54, F55F58, F63, F66, F67, F68, F69, F70, F72, F74, F76, F78, F79, F80, F81, F84, F85, F86, F93, F99, F104, F111, F112, F118, F126, F129, F130, F132, F133, F134, F135, F136,F138,F151,F158, F160, F174, F176, F177, F184, F186, F191, F197, F200, F202, F203, F207, F208, F212, F214, F217, F224, F231, F236, F238, TA50 and TA73. Such a CDR can have, for example, an amino acid sequence that is substantially similar or identical to the CDR-encoding portion of an amino acid sequence encoding one of these antibody fragments.

The present invention also provides an isolated nucleic acid molecule encoding an antibody fragment of the invention. Such a nucleic acid molecule includes a nucleotide sequence encoding substantially the amino acid sequence referenced as SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, or SEQ ID NO:136.

A nucleic acid molecule encoding an antibody fragment can include, for example, the nucleotide sequence referenced as SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, or SEQ ID NO:135.

In one embodiment, a CDR or functional fragment thereof of an antibody selective for a tumor antigen can be derived from an F3, F14, F15, F19, F21, F22, F23, F26, F133, TA50 or TA73 antibody fragment. Such a CDR can include substantially the amino acid sequence referenced as SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108,SEQ ID NO: 110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, or SEQ ID NO:136.

The present invention also provides an isolated nucleic acid molecule encoding a CDR of the invention. Such a nucleic acid molecule includes a nucleotide sequence encoding substantially the amino acid sequence referenced as SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, or SEQ ID NO:136.

A nucleic acid molecule encoding a CDR can include, for example, the nucleotide sequence referenced as SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, or SEQ ID NO:135.

It is understood that modifications which do not substantially affect the activity of the various embodiments of this invention are also included within the definition of the invention provided herein. Accordingly, the following examples are intended to illustrate but not limit the present invention.

### EXAMPLE I

### Preparation of Solid Supports Selectively Immobilizing Human Fab Fragment Populations

This example describes a method for preparing a filter for selectively immobilizing a population of binding molecules.

The population of binding molecules selectively immobilized in this example were recombinantly expressed human Fab fragments. In this case, the human BR96 Fab fragment, which is a chimeric antibody reactive with a tumor-associated cell surface antigen, was used to determine conditions for binding human Fab fragments. The BR96 antibody is reactive with a Lewis Y-related carbohydrate (LeY antigen) predominantly associated with lamp-1, an integral membrane protein normally localized in lysosomes. The population of human Fab fragments was immobilized on a solid support by coating nitrocellulose filters with anti-human kappa antibody. The anti-human kappa antibody was used as an agent for attachment to the filters since they are specific for the antibody population and can therefore be used to selectively immobilize or “capture” the population onto the filters.

Initially, conditions were determined for optimizing the amount of antibody that could be selectively immobilized on nitrocellulose filters. A variety of anti-human immunoglobulin reagents were tested as reagents for selective immobilization of human Fab fragments. Virtually all anti-human immunoglobulin reagents tested displayed enhanced signals compared to untreated nitrocellulose filters and it was determined that an affinity-purified polyclonal goat anti-human kappa antibody was most efficient at selectively immobilizing the BR96 Fab fragment. The non-specific signal generated by this approach was determined by overlaying filters both on areas not infected with phage and on phage plaques expressing an irrelevant antibody. The assay background was low and no difference was observed in the signal generated by either of these controls.

Using the polyclonal anti-human kappa antibody for selective immobilization, the concentration of anti-human kappa antibody for maximal binding of BR96 Fab fragment was determined. Briefly, molten 0.7% agar containing 1 mM isopropyl-β-D-thiogalactopyranoside (IPTG) and the supE amber suppressor strain XL1-Blue (Stratagene, San Diego, CA) was overlaid on hardened bottom 1.5%agar Luria Broth medium and incubated at 37° C. for 3 hours. A 3 μl aliquot of phage stock (˜b 10¹² phage/ml) was spotted directly on the bacterial lawn and allowed to seep into the agar for 15 minutes at 25° C. The plate was then transferred to 37° C. for 3-4 hours at which time large plaques were visible. Plaques were overlaid with 1 cm² nitrocellulose filters. The filters were removed and rinsed four times with 1.5 mM KH₂PO₄, 8 mM Na₂HPO₄, 2.7 mM KCl, 137 mM NaCl, pH 7.5 (PBS). To detect the selective immobilization of functional BR96 Fab, the filters were placed in PBS containing 0.772 μg/ml horseradish peroxidase-conjugated Lewis Y antigen, 5% nonfat powdered milk, 0.2% Tween 20, 0.01% antifoam A emulsion and 0.01% thimerosal and incubated for 2 hours at 4° C. The filters were washed five times with PBS and placed in 50 mM citric acid, 100 mM Na₂HPO₄, pH 5, containing 1 mg/ml o-phenylenediamine dihydrochloride and 0.003% hydrogen peroxide. The reaction was terminated by the addition of H₂SO₄ to a final concentration of 0.36 M, an aliquot was removed, and the OD₄₉₀ was measured in a spectrophotometer.

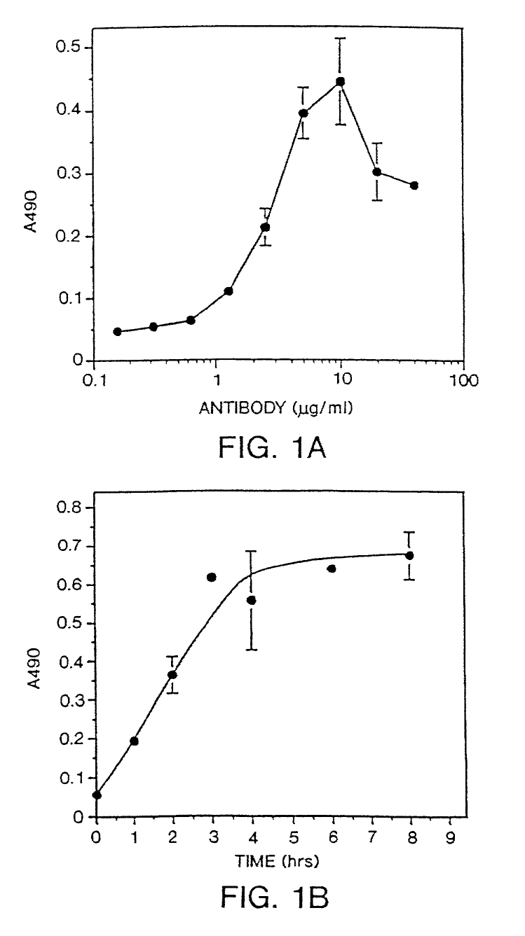

As shown in FIG. 1A, increasing concentrations of anti-human kappa antibody up to approximately 10 μg/ml retained greater quantities of BR96 Fab fragment. Filters coated with more than 10 μg/ml anti-human kappa antibody generated an enhanced, but sub-optimal signal while coating with less than 1 μg/ml antibody did not amplify the signal appreciably. Untreated filters and filters coated with low concentrations of anti-human kappa antibody (less than 0.2 μg/ml) did not generate an assay signal above background (A₄₉₀˜0.05). Therefore, based on the signal obtained from filters coated with 10 μg/ml anti-human kappa antibody (A₄₉₀˜0.45), the selective immobilization filter lift technique amplified the signal by at least nine-fold.

The amount of functional antibody Fab fragment expressed and selectively immobilized by the coated filters was assessed at various times from 0 to 8 hours at 25° C. As shown in FIG. 1B, the signal increased from 0 to 4 hours, at which point additional transfer time did not increase the signal appreciably.

Based on these results, nitrocellulose filters were incubated 3 hours in a solution containing 10 μg/ml of goat anti-human kappa antibody in PBS. To block unreacted sites on the nitrocellulose, the nitrocellulose filters were incubated 2 hours in a solution containing 1% bovine serum albumin (BSA) in PBS.

Filters prepared by this method were used in the studies described below.

### EXAMPLE II

### Immobilization of Human Fab Fragments on Solid Supports Enhanced Sensitivity for Detecting Human Fab Fragments

This example demonstrates that immobilization of binding molecule populations increases the sensitivity of detecting specific binding events.

A phage library expressing BR96-reactive Fab (240H2) was plated on E. coli bacterial lawns and grown at 37° C. for 8 hours. Control filter lifts were generated by applying nitrocellulose filters to the bacterial lawns and incubating at 22° C. for 4 hours. These control nitrocellulose filters were removed. Control filter lifts, therefore, have not selectively immobilized the binding molecules expressed in phage plaques but instead have directly immobilized all secreted polypeptides and polypeptides released by lysis, including the binding molecules. To selectively immobilize human Fab fragments, nitrocellulose filters coated with goat anti-human kappa antibody, prepared as in Example I, were applied to the bacterial lawns and incubated at 22° C for 4 hours. These selectively immobilized filter lifts were removed. Subsequent steps were carried out in parallel with control filter lifts and selectively immobilized filter lifts. Filter lifts were blocked in 1% BSA and incubated in horse radish peroxidase-conjugated BR96 antigen (LeY antigen). Filters were developed by enhanced chemiluminescence (ECL) using procedures described by the manufacturer (Amersham; Arlington Heights IL) and exposed to film for 30 seconds. The results were independent of the order the filters were applied to the bacterial lawn.

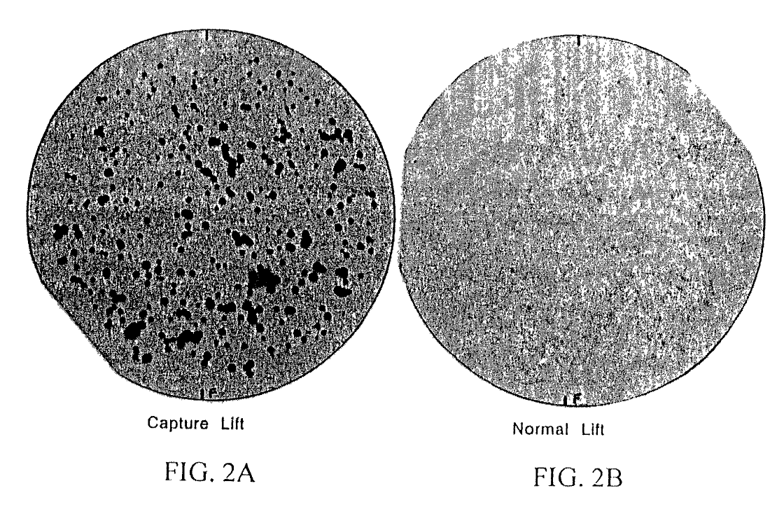

As shown in FIG. 2, human Fab fragments were readily detected on selectively immobilized filter lifts (designated capture lift in FIG. 2) whereas control filter lifts (designated normal lift in FIG. 2) had very low levels of detectable human Fab fragments. Selectively immobilized filter lifts had much stronger signals for human antibodies that recognize the tumor antigen BR96 when compared to control filter lifts where the nitrocellulose filters are applied directly to the bacterial lawn. Thus, using a selectively immobilized filter lift to increase the concentration of the specific binding molecule of interest, in this case human Fab fragments, gave greater sensitivity for detecting the binding molecule of interest.

These results show that selectively immobilized filter lifts have increased sensitivity and decreased background binding compared to control filter lifts where binding molecules are attached directly to the filter.

### EXAMPLE III

### Preparation and Extraction of Biotinylated Cell Surface Antigens

This example describes a method for labeling cell surface tumor antigens. This example also describes the preparation of cell surface tumor antigen extracts that give low background binding when used for detecting selectively immobilized antibodies.

H3396 cells were grown in monolayers and chilled on ice for subsequent manipulations at 40° C. Cells were rinsed three times with PBS. After 30 minutes, cells were incubated for 2 hours with 1.5 mg/ml sulfosuccinimidyl-6-(biotinamido)hexanoate (Pierce) in PBS. Cells were rinsed once with PBS containing 50 mM glycine followed by two more rinses with PBS. Cells were harvested by scraping in PBS containing 100 μM [4-2-aminoethyl) -benzenesulfonylfluoride, HCl], 1 AM pepstatin A and 10 μM leupeptin hemisulfate. The protein concentration was determined, Triton X-100 was added to a final concentration of 10:1 (w:w) and the suspension was incubated overnight at 4° C. on a rotating wheel. Particulate material was removed by centrifugation at 11,000×g for 30 minutes at 4° C. followed by ultracentrifugation at 540,000×g for 7 minutes 4° C. The supernatant contains solubilized biotinylated cell surface antigens.

These results demonstrate that cell surface polypeptides can be labeled with biotin for use in screening methods. These results also demonstrate that extracts devoid of interfering particulate matter, including microsomes, can be prepared for use in probing selectively immobilized filter lifts with low background binding.

### EXAMPLE IV

### Decreased Background of Selectively Immobilized Filter

Lifts Relative to Control Filter Lifts

This example demonstrates that selectively immobilized filter lifts provide decreased background binding of non-specific bacterial proteins relative to control filter lifts.

Phage expressing BR96-reactive Fab (240H2) were mixed with an irrelevant phage stock (IX64), which does not express any Fab, and plated on an E. coli bacterial lawn and grown for 8 hours at 37° C. Four replicate filter lifts (two control filter lifts and two selectively immobilized filter lifts) were incubated sequentially with the bacterial lawns for 4 hours at 22° C. Two control filter lifts (A and B) and two selectively immobilized filter lifts (C and D) were incubated with various reagents. To localize all plaques present on the bacterial lawn, filter lift A was incubated with rabbit anti-fd bacteriophage antibody in 1% BSA in PBS containing 0.1% sodium azide, washed three times in PBS, and subsequently incubated in alkaline phosphatase goat anti-rabbit antibody. Filters were washed six times with PBS and developed by incubation in alkaline phosphatase calorimetric reagent containing 0.1 M Tris, pH 9.5, 0.4 mM 2,2,′-di-p-nitrophenyl-5,5′-diphenyl-3,3′-(3,3′-dimethoxy-4,4′-diphenylene)ditetrazolium chloride and 0.38 mM 5-bromo-4-chloro-3-indoxyl phosphate mono-(p-toluidinium) salt. Filter lift C was incubated with horse radish peroxidase-conjugated Lewis Y antigen (BR96 antigen). Filter lift C was developed using ECL and exposed to film for 30 seconds.

To investigate the ability of selectively immobilized filter lifts to discriminate ligands in a cell extract, H3396 cells were biotinylated and membrane extracts were prepared as described in Example III. Filter lifts B and D were incubated for 2 hours at 4° C. with biotinylated cell surface antigens, prepared as described in Example III and diluted to 40 μg/ml tumor cell extract protein in 1% BSA, 1% Triton X-100, 0.145% sodium dodecyl sulfate and 0.1% sodium azide in PBS. Filter lifts B and D were washed six times with 0.1% Tween 20 in PBS and subsequently incubated with alkaline phosphatase-conjugated streptavidin. Plaques were visualized after developing in alkaline phosphatase calorimetric reagent.

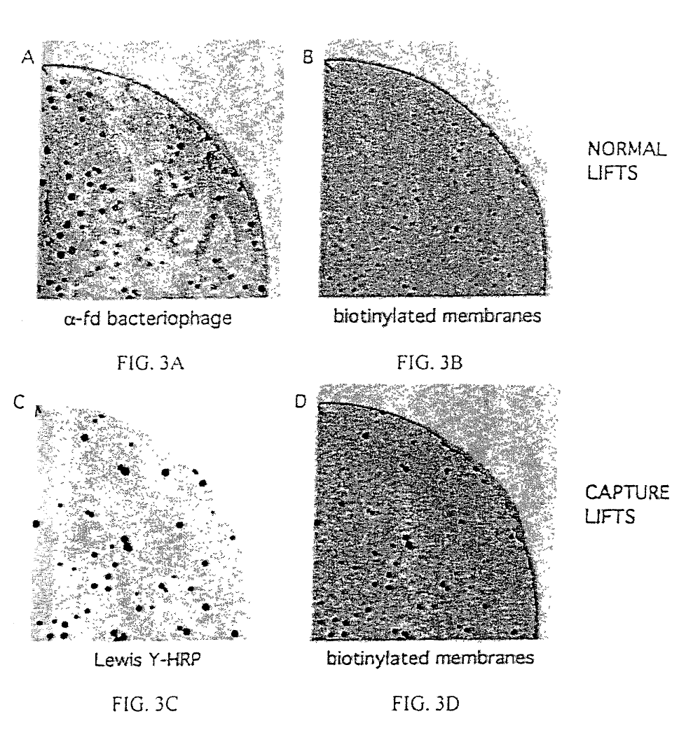

As shown in FIG. 3, all plaques were visualized with anti-fd bacteriophage antibody on filter lift A. Similarly, all plaques were visible when control lift B was probed with biotinylated cell surface antigens, even though plaques derived from IX64 do not express Fab fragments. Therefore, there appears to be significant background contributed by bacterial proteins released by phage lysis. Numerous attempts to reduce the non-specific binding of tumor cell extract were made, including: (1) utilization of a variety of detergents for the solubilization of the tumor cell membranes, (2) use of more stringent wash parameters with the filters, (3) evaluation of a wide range of blocking reagents, and (4) the use of different types of matrices to capture the phage-expressed Fab. All of these approaches either failed to reduce the background or obliterated the signal entirely.

In contrast to control filters containing directly bound binding molecules and non-specific bacterial proteins, probing selectively immobilized filter lifts with purified BR96 antigen (FIG. 3, filter lift C) or with a cell membrane lysate containing BR96 antigen as well as other biotinylated cell surface antigens (filter lift D) gave similar patterns of plaque detection. The use of crude, cell membrane lysate as source of BR96 antigen resulted in non-specific binding to all plaques, including those not expressing BR96-specific Fab fragments, on control filter lifts (filter lift B) whereas the same extract could be used to detect BR96-specific Fab fragments on selectively immobilized filter lifts (filter lift D). Therefore, selectively immobilized filter lifts had significantly lower background binding to bacterial proteins than control filter lifts.

These results indicate that selective immobilization of binding molecules to nitrocellulose filters produces decreased background binding to bacterial proteins. Therefore, capturing the binding molecule of interest by selectively immobilizing binding molecules on filters increased the concentration of the binding molecule by selectively capturing the binding molecule in the bacterial plaque lysate. In addition, background was decreased by reducing the binding of non-specific bacterial proteins. Thus, the selectively immobilized filter lift method provides enhanced sensitivity and decreased background when screening binding molecules with a population of ligands.

### EXAMPLE V

### Identification of Tumor-Specific Fab Fragments Identified by Selective Immobilization on a Solid Support

This example demonstrates the identification of clones expressing tumor-specific Fab fragments.

Fab fragment libraries were generated using breast and colon tumor tissue. A large number of Fab fragments were identified with specificity for cell surface tumor antigens.

Three libraries expressing human Fab fragments, designated A2, A3 and A4, were generated from three different tumor sources. The A2 library was constructed from tumor-draining lymph node tissue obtained from a breast cancer patient. The A3 library was constructed from human colon tumor tissue. The A4 library was constructed from prostate tumor tissue. The libraries were constructed from B cells present in the tumor tissue samples. Briefly, RNA was extracted from tumor tissue using TRIZOL reagent (Gibco-BRL, Gaithersburg, Md). Following synthesis of cDNA from the RNA, the IgG heavy and kappa light chains were amplified by touchdown polymerase chain reaction (PCR). Single-stranded DNA was isolated and cloned into two vectors, IX104 and IX203. The IX104 vector contains the amber stop codon between the immunoglobulin heavy chain and the pVIII protein. The IX203 vector has the amber stop codon removed.

The human Fab libraries were selectively immobilized on nitrocellulose filters coated with anti-human kappa antibody (prepared as in Example I). Outlined in Table II are the results of three independent experiments screening the human Fab libraries in either the IX104 or IX203 vector against biotinylated cell surface polypeptides from the H3396 cell line (prepared as described in Example III). Using the selectively immobilized filter lift technique, the frequency of positive clones varied in the three independent experiments (see Table II). As defined in these experiments, the positive clones (designated capture lift-positive clones in Table II) were identified in an initial screen, picked, replated and verified in a second selectively immobilized filter lift screen.

TABLE II
SUMMARY OF FAB LIBRARY SCREEN WITH TUMOR CELLS

| | | | Capture | | | |
|---|---|---|---|---|---|---|
| | | Random | Lift- | | Titrate on | |
| | | Clones | Positive | | Fixed H3396 | Fibroblast- |
| Library | Vector | Screened | Clones | Frequency | Monolayers | negative¹ |
| A2 | IX104 | 50,600 | 60 | 0.12% | 25 | 2+ |
| A3 | IX104 | 139,400 | 70 | 0.05% | 37 | 9+ |
| A3 | IX203 | 55,520 | 51 | 0.09% | 5 | 0 |
| A4 | IX203 | 13,000 | 22 | 0.17% | 6 | 2 |

¹unreactive at Fab fragment concentrations ≦15 μg/ml

Positive clones identified by the selectively immobilized filter lift technique were isolated and used as a source to produce greater quantities of soluble Fab for further characterization. Briefly, XL1-Blue bacteria were grown in 2X YT medium at 37° C. until the cultures reached a density of 0.9 to 1.2 OD₆₀₀. Following the addition of IPTG to a final concentration of 1 mM, the culture was infected with 10 μl of phage from a high titer stock (>10¹⁰ plaque forming units/ml) and incubated 1 hour at 37° C. The cultures were shifted to 25° C. and grown for an additional 14-16 hours. The cells were pelleted at 6000×g for 10 minutes, resuspended in 10 ml of 50 mM Tris, pH 8.0 containing 100 μM (4-(2-aminoethyl) benzenesulfonylfluoride, HCl), 1 μM pepstatin A, and 10 μM leupeptin hemisulfate, and were broken by sonic oscillation for 1 minute at 15% power output with a Branson Sonifier 450 equipped with a microtip. Following sonication, 1000 U DNaseI in 50 mM Tris, pH 8.0, was added and the lysate was incubated for 30 minutes at 25° C. The lysate was centrifuged at 12,000×g for 30 minutes at 4° C. and the supernatant was removed and used for purification of the Fab fragment.

The Fab fragment was purified using an affinity column constructed by coupling 20 mg of goat anti-human IgG Fab onto a 1 ml HiTrap NHS-activated column (Pharmacia, Piscataway, N.J.). Tween 20 was added to the bacterial cell lysate to a final concentration of 0.05% and the lysate was filtered through a 0.45 μm filter and applied to the affinity column. The unbound fraction was collected and applied a second time. The column was washed with buffer containing 50 mM Tris, pH 8.0, 0.5 M NaCl and 0.05% Tween 20 followed by 50 mM Tris, pH 8.0 buffer. The Fab was eluted with 100 mM glycine-HCl, pH 2.3, containing 0.5 M NaCl into one tenth volume 1 M Tris, pH 8.0. Fractions containing the Fab fragment were applied to a PD-10 Sephadex G25-M column (Pharmacia) which had been equilibrated in PBS.

To further characterize purified Fab fragments, H3396 cell monolayers were fixed with 2% paraformaldehyde in PBS for 15 minutes at 25° C. The paraformaldehyde was aspirated and cells were washed twice with PBS. Fixed cells were incubated in blocking buffer containing 1% BSA in PBS and Fab fragments were diluted to appropriate concentrations in blocking buffer and incubated with the fixed cells for 2 hours at 25° C. The samples were aspirated, the fixed cells washed three times with PBS and the cells were incubated with 0.5 μg/ml alkaline phosphatase-conjugated goat anti-human kappa light chain antibody for 1 hour at 25° C. The fixed cells were washed four times with PBS and developed with alkaline phosphatase calorimetric reagent.

Approximately 40-50% of the Fab fragments identified by selective immobilization on filter lifts generated a titratable signal on fixed monolayers of H3396 tumor cells, the cell line used to generate the biotinylated cell membrane lysate (see Table II). Some of the approximately 50% of Fab fragments which were not titrated on fixed monolayers are likely dependent on antigenic epitopes which were disrupted by paraformaldehyde treatment. Different techniques which do not use paraformaldehyde treatment can be used to characterize these remaining Fab fragment clones.

Positive Fab fragment clones isolated by selective immobilization on a solid support, designated capture lift-positive clones in Table II, were also tested for reactivity of the cloned Fab fragments against GM05658 normal human fibroblast cells. In one experiment where 50,600 original clones were screened, more than 2 clones did not react with fibroblast cells and were classified as fibroblast-negative. In a second experiment where 139,400 original clones were screened, more than 9 clones were identified as fibroblast-negative clones. Thus, selective immobilization allowed the identification of human Fab fragments which specifically bind to tumor cells but not normal fibroblasts.

Using the selective immobilization filter lift technique, 81 clones expressing Fab fragments were identified as shown in Table III.

DNA was isolated from Fab clones, and DNA fragments corresponding to light chain CDR regions were sequenced using the IX104 specific primer GCCAGTTCCAGATTTCAACTG (SEQ ID NO:1) or the IX203 specific primer CTCTGTGACACTCTCCTGGGA (SEQ ID NO:2). DNA fragments corresponding to heavy chain CDR regions were sequenced using primer GTAGTCCTTGACCAGGCA (SEQ ID NO:3) or AAGACCGATGGGCCCTTGGTGGAGGC (SEQ ID NO:4). Table IV shows the amino acid sequences of three CDR regions found in the light chain of the cloned Fab fragments. Table V shows the nucleotide sequences of the corresponding CDR regions of the light chain of cloned Fab fragments. Table VI shows the amino acid sequences of three CDR

TABLE III
Tumor-Specific Human Fab Clones Identified by
the Selective Immobilization Filter Lift Technique

| clone | library | family |
|---|---|---|
| F3 | A3.104 | VH3 |
| F13 | A3.104 | VH4 |
| F14 | A3.104 | VH3 |
| F15 | A3.104 | VH3, VK3 |
| F19 | A3.104 | VH4 |
| F21 | A3.104 | VH1 |
| F22 | A3.104 | VH6 |
| F23 | A3.104 | VH3, VK1 |
| F26 | A3.104 | VH3 |
| F30 | A2.104 | |
| F31 | A2.104 | |
| F32 | A2.104 | |
| F33 | A2.104 | |
| F34 | A3.104 | |
| F35 | A3.104 | |
| F36 | A3.104 | |
| F37 | A3.104 | |
| F38 | A3.104 | |
| F40 | A2.104 | |
| F41 | A2.104 | |
| F42 | A2.104 | |
| F46 | A2.104 | |
| F49 | A2.104 | |
| F50 | A2.104 | |
| F52 | A2.104 | |
| F54 | A2.104 | |
| F55 | A2.104 | |
| F58 | A2.104 | |
| F63 | A3.104 | |
| F66 | A3.104 | |
| F67 | A3.104 | |
| F68 | A3.104 | |
| F69 | A3.104 | |
| F70 | A3.104 | |
| F72 | A3.104 | |
| F74 | A3.104 | |
| F76 | A3.104 | |
| F78 | A3.104 | |
| F79 | A3.104 | |
| F80 | A3.104 | |
| F81 | A3.104 | |
| F84 | A3.104 | |
| F85 | A3.104 | |
| F86 | A3.104 | VH3, VK1 |
| F93 | A3.203 | |
| F99 | A3.104 | |
| F104 | A3.104 | |
| F111 | A3.104 | |
| F112 | A3.104 | |
| F118 | A3.104 | |
| F126 | A3.203 | |
| F129 | A3.203 | |
| F130 | A3.203 | |
| F132 | A3.104 | VH3, VK1 |
| F133 | A3.104 | VH3, VK1 |
| F134 | A3.104 | VH3, VK1 |
| F135 | A3.104 | VH3, VK1 |
| F136 | A3.104 | |
| F138 | A3.203 | |
| F151 | A3.203 | |
| F158 | A3.203 | |
| F160 | A3.203 | |
| F174 | A3.203 | |
| F176 | A3.203 | |
| F177 | A4.203 | |
| F184 | A4.203 | |
| F186 | A4.203 | |
| F191 | A4.203 | |
| F197 | A4.203 | |
| F200 | A4.203 | |
| F202 | A4.203 | |
| F203 | A4.203 | |
| F207 | A4.203 | |
| F208 | A4.203 | |
| F212 | A4.203 | |
| F214 | A4.203 | |
| F217 | A4.203 | |
| F224 | A4.203 | |
| F231 | A4.203 | |
| F236 | A4.203 | |
| F238 | A4.203 | |
| TA50 | A3.104 | VH5, VK3 |
| TA73 | A3.104 | VH1, VK1 |

regions found in the heavy chain of the cloned Fab fragments. Table VII shows the nucleotide sequences of the corresponding CDR regions of the heavy chain of cloned Fab fragments. Each of the Fab clones identified by selective immobilization on filters contains nucleotide sequence encoding unique amino acid sequence.

TABLE IV
Amino Acid Sequences of CDR Regions in the
Light Chains of Fab Fragments Isolated by the
Selective Immobilization Filter Lift Technique

Clone

Light Chain CDR 1

| | | |
|---|---|---|
| F3 | Q A S H S V F N L V G | SEQ ID NO:6 |
| F14 | R D H S N V L A A A S W A | SEQ ID NO:8 |
| F15 | R A S Q R V S S R F V A | SEQ ID NO:10 |
| F19 | R G L V R T L N N H L N | SEQ ID NO:12 |
| F21 | R A S Q S V S S T N L T | SEQ ID NO:14 |
| F22 | R A K Q N I G R W L A | SEQ ID NO:16 |
| F23 | R A S Q D I N T Y L A | SEQ ID NO:18 |
| F26 | R A V Q R I K Q N F G N | SEQ ID NO:20 |
| F133 | R A S Q D I N T Y L A | SEQ ID NO:22 |
| TA50 | R A S Q T V R S S Y L A | SEQ ID NO:24 |
| TA73 | Q A N E D T G H R L N | SEQ ID NO:26 |

Light Chain CDR 2

| | | |
|---|---|---|
| F3 | K S S N F T K | SEQ ID NO:28 |
| F14 | G A S T R A T | SEQ ID NO:30 |
| F15 | G A S S R A S | SEQ ID NO:32 |
| F19 | A A S R L Q S | SEQ ID NO:34 |
| F21 | A A S T L Q T | SEQ ID NO:36 |
| F22 | R A S S L E S | SEQ ID NO:38 |
| F23 | R A S T L D I | SEQ ID NO:40 |
| F26 | K A S R L E N | SEQ ID NO:42 |
| F133 | R A S T L D I | SEQ ID NO:44 |
| TA50 | G A S I R A P | SEQ ID NO:46 |
| TA73 | R S S N L K K | SEQ ID NO:48 |

Light Chain CDR 3

| | | |
|---|---|---|
| F3 | Q Q Y S S T F G T | SEQ ID NO:50 |
| F14 | Q H Y K S A P P R H T | SEQ ID NO:52 |
| F15 | Q Q S H R T P Y T | SEQ ID NO:54 |
| F19 | Q Q S S V S R T | SEQ ID NO:56 |
| F21 | Q H A Y A F P R T | SEQ ID NO:58 |
| F22 | Q H Y N T N S P L T | SEQ ID NO:60 |
| F23 | Q Q Y S R Y P L T | SEQ ID NO:62 |
| F26 | Q L Y D S H S A M Y T | SEQ ID NO:64 |
| F133 | Q Q Y S R Y P L T | SEQ ID NO:66 |
| TA50 | Q Q Y G R S P R A | SEQ ID NO:68 |
| TA73 | L Q D F N Y P R T | SEQ ID NO:70 |

TABLE V
Nucleotide Sequences of CDR Regions in the
Light Chains of Fab Fragments Isolated by the
Selective Immobilization Filter Lift Technique

Clone

Light Chain CDR 1

| | | |
|---|---|---|
| F3 | CAG GCC AGT CAC AGT GTT TTT AAC | SEQ ID NO:5 |
| | TTG GTT GGC | |
| F14 | AGG GAC CAT TCA AAC GTG TTA GCA | SEQ ID NO:7 |
| | GCA GCT TCA TGG GCC | |
| F15 | AGG GCC AGT GAG CGT GTT AGC AGC | SEQ ID NO:9 |
| | AGA TTC GTA GCT | |
| F19 | CGG GGG CTA GTC AGG ACA TTA AAC | SEQ ID NO:11 |
| | AAC CAT CTA AAT | |
| F21 | AGG GCC AGT CAG AGT GTT AGC AGC | SEQ ID NO:13 |
| | ACT AAC TTA ACT | |
| F22 | CGG GCC AAG CAG AAT ATT GGT CGG | SEQ ID NO:15 |
| | TGG TTG GCT | |
| F23 | CGG GCC AGT CAG GAC ATT AAC ACT | SEQ ID NO:17 |
| | TAT TTA GCC | |
| F26 | CGG GCA GTT CAA CGC ATT AAA CAA | SEQ ID NO:19 |
| | AAT TTT GGG AAT | |
| F133 | CGG GCC AGT CAG GAC ATT AAC ACT | SEQ ID NO:21 |
| | TAT TTA GCC | |
| TA50 | AGG GCC AGT CAG ACT GTT AGG TCC | SEQ ID NO:23 |
| | AGC TAC TTA GCC | |
| TA73 | CAG GCG AAT GAG GAC ATT GGC CAC | SEQ ID NO:25 |
| | CGT TTA AAT | |

Light Chain CDR 2

| | | |
|---|---|---|
| F3 | AAG TCA TCT AAC TTT ACA AAG | SEQ ID NO:27 |
| F14 | GGT GCA TCC ACC AGG GCC ACC | SEQ ID NO:29 |
| F15 | GGT GCA TCC AGC AGG GCT TCT | SEQ ID NO:31 |
| F19 | GCT GCG TCC CGC TTG CAA AGT | SEQ ID NO:33 |
| F21 | GCT GCA TCC ACT TTG CAA ACT | SEQ ID NO:35 |
| F22 | AGG GCG TCC AGT TTA GAA AGT | SEQ ID NO:37 |
| F23 | AGG GCG TCT ACT TTA GAC ATT | SEQ ID NO:39 |
| F26 | AAG GCG TCT CGT TTA GAA AAT | SEQ ID NO:41 |
| F133 | AGG GCG TCT ACT TTA GAC ATT | SEQ ID NO:43 |
| TA50 | GGT GCA TCC ATC AGG GCC CCT | SEQ ID NO:45 |
| TA73 | CGT TCA TCC AAT TTG AAA AAA | SEQ ID NO:47 |

Light Chain CDR 3

| | | |
|---|---|---|
| F3 | CAA CAG TAT TCG AGT ACT TTC GGG | SEQ ID NO:49 |
| | ACC | |
| F14 | CAG CAT TAT AAG AGC GCA CCT CCG | SEQ ID NO:51 |
| | AGG CAC ACT | |
| F15 | CAA CAG AGT CAT AGA ACC CCG TAC | SEQ ID NO:53 |
| | ACT | |
| F19 | CAA CAG AGT TCC GTT AGT CGG ACG | SEQ ID NO:55 |
| F21 | CAG CAC GCT TAC GCT TTC CCT CGG | SEQ ID NO:57 |
| | ACG | |
| F22 | CAG CAC TAT AAC ACT AAC TCT CCG | SEQ ID NO:59 |
| | CTC ACT | |
| F23 | CAA CAG TAT AGT CGT TAT CCG CTC | SEQ ID NO:61 |
| | ACT | |
| F26 | CAA CTA TAT GAT AGT CAT TCC GCG | SEQ ID NO:63 |
| | ATG TAC ACT | |
| F133 | CAA CAG TAT AGT CGT TAT CCG CTC | SEQ ID NO:65 |
| | ACT | |
| TA50 | CAG CAG TAT GGT AGG TCA CCT CGG | SEQ ID NO:67 |
| | GCG | |
| TA73 | CTG CAG GAT TTC AAT TAT CCC CGG | SEQ ID NO:69 |
| | ACG | |

TABLE VI
Amino Acid Sequences of CDR Regions in the
Heavy Chains of Fab Fragments Isolated by the
Selective Immobilization Filter Lift Technique

Clone

Heavy Chain CDR 1

| | | |
|---|---|---|
| F3 | S Y S M N | SEQ ID |
| | | NO:72 |
| F14 | D Y A I H | SEQ ID |
| | | NO:74 |
| F15 | T Y W M S | SEQ ID |
| | | NO:76 |
| F19 | S S L W W S | SEQ ID |
| | | NO:78 |
| F21 | R Y Y M N | SEQ ID |
| | | NO:80 |
| F22 | S N R A T W N | SEQ ID |
| | | NO:82 |
| F23 | S Y A M N | SEQ ID |
| | | NO:84 |
| F26 | F S N Y D L N | SEQ ID |
| | | NO:86 |
| F133 | S A W L S | SEQ ID |
| | | NO:88 |
| TA50 | S Y W I S | SEQ ID |
| | | NO:90 |
| TA73 | T Y G L S | SEQ ID |
| | | NO:92 |

Heavy Chain CDR 2

| | | |
|---|---|---|
| F3 | Y F S S S G T T I Y Y A D S V K G | SEQ ID |
| | | NO:94 |
| F14 | F I R S E A F G G T A D Y A A S V K G | SEQ ID |
| | | NO:96 |
| F15 | N I N Q D G S E K Y Y V D S V E G | SEQ ID |
| | | NO:98 |
| F19 | E M Y H S G S T K Y N P P S R V | SEQ ID |
| | | NO:100 |
| F21 | I I N P S G D R T R Y A Q K F Q G | SEQ ID |
| | | NO:102 |
| F22 | R T Y Y R S E W F N D Y A V F V Q S | SEQ ID |
| | | NO:104 |
| F23 | Y I S S R G S T I Y Y A D S V K G | SEQ ID |
| | | NO:106 |
| F26 | T F T G S G D N T Y Y A D S V K G | SEQ ID |
| | | NO:108 |
| F133 | N I K P D G S D K Y Y V D S V K G | SEQ ID |
| | | NO:110 |
| TA50 | R I D P S D S Y T N Y S P S F Q G | SEQ ID |
| | | NO:112 |
| TA73 | W I K P Y N G Y T K Y A Q R L Q D | SEQ ID |
| | | NO:114 |

Heavy Chain CDR 3

| | | |
|---|---|---|
| F3 | G R K T S R Y T L R R F R S I W F D Y | SEQ ID |
| | | NO:116 |
| F14 | V G R P G E D F | SEQ ID |
| | | NO:118 |
| F15 | D P P R Y F G S G S W E | SEQ ID |
| | | NO:120 |
| F19 | A S Y T N K W T P I A Y F E Y | SEQ ID |
| | | NO:122 |
| F21 | P L V Q R D T F Q E | SEQ ID |
| | | NO:124 |
| F22 | V P Q V E V P Y | SEQ ID |
| | | NO:126 |
| F23 | F M N R D G P P K R V F D Y | SEQ ID |
| | | NO:128 |
| F26 | G V S T M V R G L T | SEQ ID |
| | | NO:130 |
| F133 | A G P L R I R Y D G F D F | SEQ ID |
| | | NO:132 |
| TA50 | G R K T S R Y T L R R S E A S G F D Y | SEQ ID |
| | | NO:134 |
| TA73 | D A H R G L V G A T A N F H Y | SEQ ID |
| | | NO:136 |

TABLE VII
Nucleotide Sequences of CDR Regions in the
Heavy Chains of Fab Fragments Isolated by the
Selective Immobilization Filter Lift Technique

Clone

Heavy Chain CDR 1

| | | |
|---|---|---|
| F3 | TCC TAT AGC ATG AAC | SEQ ID NO:71 |
| F14 | GAC TAT GCT ATA CAT | SEQ ID NO:73 |
| F15 | ACC TAT TGG ATG AGC | SEQ ID NO:75 |
| F19 | AGT CTT TGG TGG AGT | SEQ ID NO:77 |
| F21 | AGG TAT TAT ATG AAC | SEQ ID NO:79 |
| F22 | AGC AAT CGT GCT ACG TGG AAC | SEQ ID NO:81 |
| F23 | AGC TAT GCC ATG AAT | SEQ ID NO:83 |
| F26 | TTT AGT AAT TAT GAT CTG AAC | SEQ ID NO:85 |
| F133 | AGC GCC TGG CTG AGC | SEQ ID NO:87 |
| TA50 | AGC TAC TGG ATC AGC | SEQ ID NO:89 |
| TA73 | ACG TAT GGT CTC AGC | SEQ ID NO:91 |

Heavy Chain GDR 2

| | | |
|---|---|---|
| F3 | TAT TTT AGT AGT AGT GGG ACC ACC | SEQ ID NO:93 |
| | ATA TAT TAC GCA GAC TCT GTG AAG | |
| | GGC | |
| F14 | TTG ATT AGA AGT GAG GCC TTT GGT | SEQ ID NO:95 |
| | GGG ACA GCA GAT TAC GCC GCG TCT | |
| | GTG AAA GGC | |
| F15 | AAC ATA AAC CAA GAT GGA AGT GAG | SEQ ID NO:97 |
| | AAA TAC TAT GTG GAC TCT GTG GAG | |
| | GGC | |
| F19 | GAA ATG TAT CAT AGT GGG AGC ACC | SEQ ID NO:99 |
| | AAA TAC AAC CCT CCC TCA AGA GTT | |
| F21 | ATT ATC AAC CCT AGT GGT GAT AGA | SEQ ID NO:101 |
| | ACA AGA TAC GCA CAG AAG TTC CAG | |
| | GGC | |
| F22 | AGG ACA TAC TAC AGG TCC GAG TGG | SEQ ID NO:103 |
| | TTT AAT GAT TAT GCA GTA TTT GTG | |
| | CAA AGT | |
| F23 | TAC ATT AGT AGT CGT GGT AGT ACC | SEQ ID NO:105 |
| | ATA TAC TAC GCT GAC TCT GTG AAG | |
| | GGC | |
| F26 | ACT TTT ACT GGT AGT GGT GAC AAC | SEQ ID NO:107 |
| | ACA TAC TAC GCA GAC TCC GTG AAG | |
| | GGC | |
| F133 | AAC ATA AAG CCA GAT GGA AGT GAC | SEQ ID NO:109 |
| | AAA TAT TAT GTG GAC TCT GTG AAG | |
| | GGC | |
| TA50 | AGG ATT GAT CCT AGT GAC TCT TAT | SEQ ID NO:111 |
| | ACC AAC TAC AGC CCG TCC TTC CAA | |
| | GGC | |
| TA73 | TGG ATC AAG CCT TAC AAT GGT TAT | SEQ ID NO:113 |
| | ACA AAG TAT GCA CAG CGC CTC CAG | |
| | GAT | |

Heavy Chain CDR 3

| | | |
|---|---|---|
| F3 | GGA AGG AAG ACC TCT CGA TAT ACT | SEQ ID NO:115 |
| | TTG CGA CGT TTC AGA AGT ATC TGG | |
| | TTT GAC TAC | |
| F14 | GTC GGG CGC CCG GGT GAG GAC TTT | SEQ ID NO:117 |
| F15 | GAT CCC CCA CGA TAC TTT GGT TCG | SEQ ID NO:119 |
| | GGG AGT TGG GAA | |
| F19 | GCC AGT TAT ACC AAC AAA TGG ACC | SEQ ID NO:121 |
| | CCC ATT GCC TAC TTT GAA TAC | |
| F21 | CCC CTG GTT CAA CGG GAT ACT TTC | SEQ ID NO:123 |
| | CAG GAA | |
| F22 | GTC CCA CAG GTA GAG GTG CCT TAT | SEQ ID NO:125 |
| F23 | TTT ATG AAT AGA GAT GGC CCC CCG | SEQ ID NO:127 |
| | AAG AGG GTG TTT GAC TAC | |
| F26 | GGG GTT TCG ACT ATG GTT CGG GGA | SEQ ID NO:129 |
| | CTG ACC | |
| F133 | GCT GGC CCA CTC CGT ATC CGG TAT | SEQ ID NO:131 |
| | GAT GGT TTT GAT TTT | |
| TA50 | GGA AGG AAG ACC TCT CGA TAT ACT | SEQ ID NO:133 |
| | TTG CGA CGT TCA GAA GCA TCT GGT | |
| | TTC GAC TAC | |
| TA73 | GAT GCC CAT CGC GGT CTA GTG GGA | SEQ ID NO:135 |
| | GCT ACA GCA AAC TTT CAC TAC | |

Using the selective immobilization filter lift technique, a large number of human Fab fragments produced in an expression library were identified. The technique allowed the screening of a large number of clones. DNA sequencing of selected numbers of isolated Fab clones revealed that a variety of genetically distinct Fab fragments were identified.

These results show that the selective immobilization filter lift technique allows identification of a large number of distinct human Fab fragments.

### EXAMPLE VI

Characterization of Tumor Cell Specific Human Fab Fragments Identified by Selective Immobilization on a Solid Support

This example demonstrates cell specific reactivity of Fab clones with respect to fixed and live tumor cell lines.

Fab fragments identified using the selective immobilization filter lift technique were purified as described in Example V. Various cell lines were fixed in paraformaldehyde as described in Example V and incubated with increasing concentrations of purified Fab fragments (2.5×10⁻¹ to 3×10¹ μg/ml). Unbound Fab was removed and fixed cells were incubated with alkaline phosphatase-conjugated anti-human kappa antibody as described in Example V. Following incubation with color reagents, the OD₅₆₀ was measured.

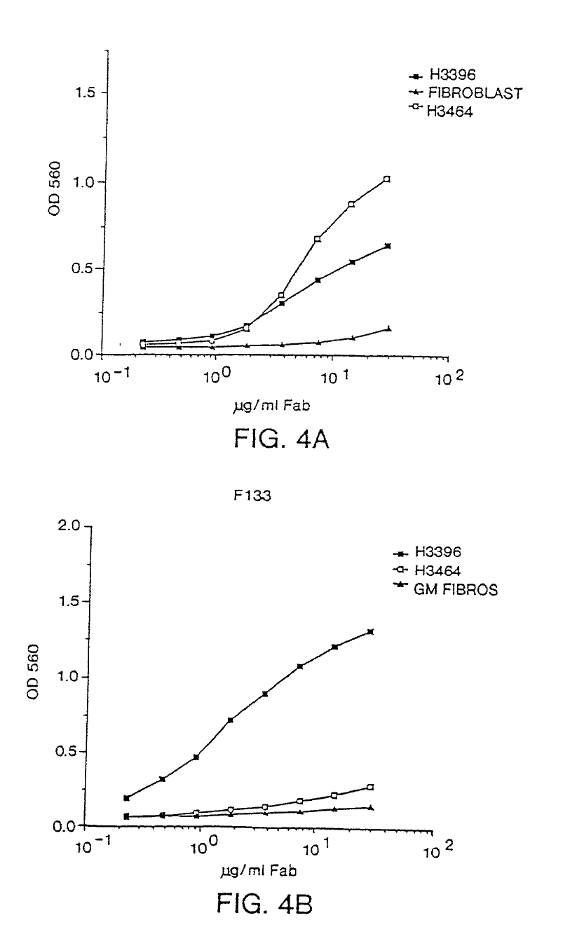

In FIG. 4A, the titration curve of purified Fab F15 is shown on two breast carcinoma cell lines (H3396 and H3464) and GMO5658 normal human fibroblasts. Fab F15 shows specific binding to both breast cell lines but not to normal fibroblasts. In FIG. 4B, the titration curve of Fab F133 is shown with the same two breast cell lines and GM05658 normal human fibroblasts. Fab F133 shows specific binding for the H3396 cells used for making a screening extract. However, Fab F133 does not bind to the second breast cell line or to the normal fibroblasts.

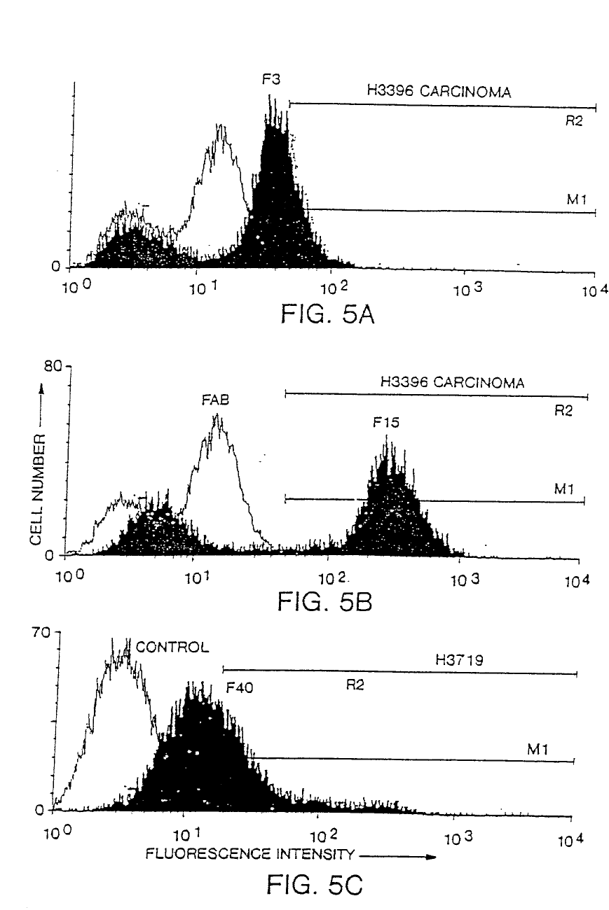

Purified Fab fragments were also characterized with respect to binding to live tumor cells by FACS analysis on a Becton Dickinson FACSort system. Purified Fab F3 was compared to a non-specific Fab fragment on H3396 cells (see FIG. 5A). Incubation of H3396 cells with Fab F3 caused a shift in H3396 cells, indicating reactivity with a cell surface antigen, compared to cells incubated with non-specific Fab. The peak of Fab F3 shifted cells is sharp, indicating that the population of H3396 cells are relatively homogeneous in their expression of antigen.

A similar FACS analysis was performed with Fab F15. As shown in FIG. 5B, Fab F15 resulted in a larger shift in H3396 cells relative to non-specific Fab than was observed with Fab F3, indicating that Fab F15 reacts with a cell surface antigen. The Fab F15 shifted peak is broader than that observed with Fab F3, consistent with a more heterogeneous expression of the Fab F15 antigen on H3396 cells.

A FACS analysis was also performed with Fab F40. FAB F40 was identified using membrane extracts of the breast cell line H3396. Interestingly, Fab F40 is cross-reactive with the colon carcinoma cell line H3719 (see FIG. 5C). Fab F40 results in a shift in H3719 cells relative to control non-specific Fab, indicating that Fab F40 recognizes a cell surface antigen on the colon carcinoma cell line. The Fab F40 peak is somewhat broad, indicating that expression of the F40 antigen is more heterogeneous. Thus, Fab F40 recognizes a cell surface antigen on two different types of tumor cells.

The selective immobilization filter lift technique allowed isolation of a large number of human Fab fragments with specificity for fixed and live tumor cells. While some Fab fragments are specific for breast carcinoma cells or are specific for a particular breast cell line, other Fab fragments cross react with a colon carcinoma cell line. These Fab fragments displayed specific binding on tumor cells but did not bind to normal fibroblasts.

These results indicate that the selective immobilization filter lift technique allows identification of human Fab fragments with specificity for tumor cells.

All journal article and references provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

1. A method comprising:
a. providing:
i. a solid support coated with an anti-immunoglobulin reagent; and
ii. a phage expressed antibody library; and
b. contacting said solid support to said phage expressed antibody library.

2. The method of claim 1, wherein said contacting generates an antibody bound solid support.

3. The method of claim 2, further comprising the step of c) contacting said antibody bound solid support with a sample containing antigens.

4. The method of claim 3, wherein said contacting step of step c) generates a solid support containing antibody-antigen complexes.

5. The method of claim 4, further comprising the step of d) identifying one or more antigens contained in said antibody-antigen complexes.

6. The method of claim 4, further comprising the step of e) generating an immunoglobulin molecule that binds at least one antigen found in said antibody-antigen complexes.

7. The method of claim 6, further comprising the step of f) treating a cell with said immunoglobulin.

8. The method of claim 7, wherein said cell comprises a cancer cell.

9. The method of claim 3, wherein said sample comprises a cell extract.

10. The method of claim 9, wherein said cell extract comprises a cancer cell extract.

11. The method of claim 10, wherein said cancer cell extract contains biotinylated proteins.

12. The method of claim 11, wherein said biotinylated proteins comprise biotinylated membrane proteins.

13. The method of claim 2, further comprising the step of contacting said antibody bound solid support with a label.

14. The method of claim 1, wherein said phage expressed antibody library expresses antibody fragments.

15. The method of claim 14, wherein said antibody fragments comprise antibody fragments reactive with surface expressed cancer polypeptides.

16. The method of claim 1, wherein said solid support comprises a membrane.

17. The method of claim 16, wherein said membrane comprises a nitrocellulose membrane.

18. The method of claim 1, wherein said solid surface is further coated with a blocking agent.

19. The method of claim 18, wherein said blocking agent comprises bovine serum albumin.

20. The method of claim 1, wherein said anti-immunoglobulin reagent comprises anti-human antibody.

21. The method of claim 20, wherein said anti-human antibody comprises anti-human kappa antibody.

22. The method of claim 21, wherein said anti-human kappa antibody comprises goat anti-human kappa antibody.

23. The method of claim 2, wherein said antibody bound solid support comprises more bound antibody than a control solid support lacking said anti-immunoglobulin reagent.

24. The method of claim 18, wherein said contacted solid support binds less non-human protein than a control support without said blocking agent.

25. A method comprising:
a) providing:
i) a solid support comprising anti-immunoglobulin bound to antibodies derived from an expression library; and
ii) a cell extract; and
b) contacting said solid support and said cell extract, wherein said contacting results in binding of antigens from said cell extract to said antibodies.

26. The method of claim 25, wherein said cell extract comprises a detergent solubilized cell extract.

27. The method of claim 25, further comprising the step of c) identifying one or more antigens bound to said antibodies.

28. The method of claim 27, further comprising the step of d) generating an immunoglobulin molecule that binds at least one of said identified antigens.

29. The method of claim 28, further comprising the step of e) treating a cell with said immunoglobulin.

30. The method of claim 29, wherein said cell comprises a cancer cell.

31. The method of claim 25, wherein said cell extract comprises a cancer cell extract.

32. The method of claim 31, wherein said cancer cell extract contains biotinylated proteins.

33. The method of claim 32, wherein said biotinylated proteins comprise biotinylated membrane proteins.

34. The method of claim 25, wherein said solid support comprises a membrane.

35. The method of claim 34, wherein said membrane comprises a nitrocellulose membrane.

36. The method of claim 25, wherein said solid surface is further coated with a blocking agent.

37. The method of claim 36, wherein said blocking agent comprises bovine serum albumin.

38. The method of claim 1, wherein said anti-immunoglobulin reagent comprises anti-human antibody.

39. The method of claim 38, wherein said anti-human antibody comprises anti-human kappa antibody.

40. The method of claim 39, wherein said anti-human kappa antibody comprises goat anti-human kappa antibody.

41. A method comprising:
a. providing:
i. a solid support coated with a reagent; and
ii. an expressed antibody library; and
b. contacting said solid support to said expressed antibody library under conditions such that a reagent/antibody complex is formed.

42. The method of claim 41, wherein antibody in said reagent/antibody complex has an antigen binding site available for binding to an antigen.

43. The method of claim 41, wherein said reagent comprises an anti-immunoglobulin reagent.

44. The method of claim 41, wherein said reagent comprises a peptide.

45. The method of claim 41, wherein said expressed antibody library comprises a phage expressed antibody library.

46. The method of claim 41, wherein said expressed antibody library comprises a hybridoma lysate.

47. The method of claim 41, wherein said expressed antibody library comprises a bacteria expressed antibody library.

48. The method of claim 41, further comprising the step of c) contacting said reagent/antibody complex with a sample containing antigens.

49. The method of claim 48, wherein said contacting step of step c) generates an antibody-antigen complex.

50. The method of claim 49, further comprising the step of d) identifying one or more antigens contained in said antibody-antigen complex.

51.The method of claim 50, further comprising the step of e) generating an immunoglobulin molecule that binds at least one antigen found in said antibody-antigen complex.

52. The method of claim 51, further comprising the step of f) treating a cell with said immunoglobulin.

53. The method of claim 52, wherein said cell comprises a cancer cell.

54. The method of claim 48, wherein said sample comprises a cell extract.

55. The method of claim 54, wherein said cell extract comprises a cancer cell extract.

56. The method of claim 55, wherein said cancer cell extract contains biotinylated proteins.

57. The method of claim 56, wherein said biotinylated proteins comprise biotinylated membrane proteins.

58. The method of claim 41, wherein said expressed antibody library comprises expressed antibody fragments.

59. The method of claim 58, wherein said antibody fragments comprise antibody fragments reactive with surface expressed cancer polypeptides.

60. The method of claim 41, wherein said solid support comprises a membrane.

61. The method of claim 60, wherein said membrane comprises a nitrocellulose membrane.

62. The method of claim 41, wherein said solid surface is further coated with a blocking agent.

63. The method of claim 62, wherein said blocking agent comprises bovine serum albumin.

64. The method of claim 43, wherein said anti-immunoglobulin reagent comprises anti-human antibody.

65. The method of claim 64, wherein said anti-human antibody comprises anti-human kappa antibody.

66. The method of claim 65, wherein said anti-human kappa antibody comprises goat anti-human kappa antibody.

67. The method of claim 41, wherein said solid support comprises more reagent/antibody complex than a control solid support lacking said reagent.

68. The method of claim 62, wherein said solid support binds less non-human protein than a control support without said blocking agent.

69. A method comprising:
a. providing:
i. a solid support coated with a reagent; and
b. contacting said solid support to said expressed protein library under conditions such that a reagent/protein complex is formed.

70. The method of claim 69, wherein said reagent comprises a peptide.

71. The method of claim 69, wherein said expressed protein library comprises a bacteria expressed protein library.

72. The method of claim 71, wherein said bacteria expressed protein library comprises an antibody library.

73. The method of claim 69, wherein said expressed protein library comprises a phage expressed protein library.

74. The method of claim 74, wherein said phage expressed library comprises an antibody library.

75. The method of claim 69, further comprising the step of c) contacting said bound protein complex with a sample containing ligands.

76. The method of claim 75, wherein said contacting step of step c) generates a protein-ligand complex.

77. The method of claim 76, further comprising the step of d) identifying one or more ligands contained in said protein-ligand complex.

78. The method of claim 77, further comprising the step of e) generating an immunoglobulin molecule that binds at least one ligand found in said protein-ligand complex.

79. The method of claim 78, further comprising the step of f) treating a cell with said immunoglobulin.

80. The method of claim 79, wherein said cell comprises a cancer cell.

81. The method of claim 75, wherein said sample comprises a cell extract.

82. The method of claim 81, wherein said cell extract comprises a cancer cell extract.

83. The method of claim 82, wherein said cancer cell extract contains biotinylated proteins.

84. The method of claim 83, wherein said biotinylated proteins comprise biotinylated membrane proteins.

85. The method of claim 69, wherein said expressed protein library comprises protein fragments.

86. The method of claim 85, wherein said protein fragments comprise protein fragments reactive with surface expressed cancer polypeptides.

87. The method of claim 69, wherein said solid support comprises a membrane.

88. The method of claim 87, wherein said membrane comprises a nitrocellulose membrane.

89. The method of claim 69, wherein said solid surface is further coated with a blocking agent.

90. The method of claim 89, wherein said blocking agent comprises bovine serum albumin.

91. The method of claim 69, wherein said solid support comprises more bound protein complex than a control support lacking said reagent.
